(19) United States
(12) Patent Application Publication
(10) Pub. No.: US 2025/0302767 A1
(43) Pub. Date: Oct. 2, 2025

(54) TREATMENT OF CANCER AND AUTOIMMUNE DISORDERS USING NANO POLYMERS OF HISTONE DEACETYLASE INHIBITORS

(71) Applicant: University of Virginia Patent Foundation, Charlottesville, VA (US)

(72) Inventors: Mark Kester, Afton, VA (US); David J. Feith, Palmyra, VA (US); Anuradha Illendula, Crozet, VA (US); Thomas P. Loughran, JR., Charlottesville, VA (US); John Sanil Manavalan, Charlottesville, VA (US); Enrica Marchi, Charlottesville, VA (US); Owen O’Connor, Scarsdale, NY (US); Ipsita Pal, Charlottesville, VA (US)

(73) Assignee: University of Virginia Patent Foundation, Charlottesville, VA (US)

(21) Appl. No.: 18/701,581

(22) PCT Filed: Oct. 17, 2022
(86) PCT No.: PCT/US2022/046905

§ 371 Date: Apr. 15, 2024

Related U.S. Application Data
(60) Provisional application No. 63/256,246, filed on Oct. 15, 2021.

(51) Int. Cl. A61K 9/51; A61K 31/167; A61K 31/18; A61K 31/4045; A61K 38/15; A61K 45/06; A61P 35/00; A61P 35/02

(52) U.S. Cl. CPC A61K 9/5153; A61K 9/5192; A61K 31/167; A61K 31/18; A61K 31/4045; A61K 38/15; A61K 45/06; A61P 35/00; A61P 35/02

(57) ABSTRACT

Provided are compositions that include a histone deacetylase inhibitor (HDACi) encapsulated in and/or otherwise associated with a nanoparticle. In some embodiments, the HDACi is romidepsin, vorinostat, belinostat, panobinostat, and/or chidamide. In some embodiments, the nanoparticle is a poly(D,L-lactide)-PEG-methyl ether (mPEG-PDLLA) nanopolymer. Also provided are methods for treating diseases, disorders, and/or conditions associated with sensitivity to histone deacetylase inhibitors, such as but not limited to tumors and/or cancers; and methods for inhibiting the growth, proliferation, and/or metastasis of a tumor and/or a cancer associated with sensitivity to histone deacetylase inhibitors by administering an effective amount of a composition as disclosed herein, which methods can optionally include administering at least one additional therapeutically active agent, such as but not limited to a chemotherapeutic agent.

### CROSS-REFERENCE TO RELATED APPLICATION

This application claims priority to and the benefit of U.S. Provisional Patent Application Ser. No. 63/256,246, filed Oct. 15, 2021, the entire disclosure of which is herein incorporated by reference.

### TECHNICAL FIELD

The presently disclosed subject matter relates in some embodiments to methods for treating diseases, disorders, and conditions with histone deacetylase inhibitors (HDACi), particularly romidepsin. In some embodiments, the disease, disorder, or condition is a tumor and/or a cancer.

### BACKGROUND

Romidepsin ((1S,4S,7Z,10S,16E,21R)-7-Ethylidene-4,21-diisopropyl-2-oxa-12,13-dithia-5,8,20,23-tetrazabicyclo[8.7.6]tricos-16-ene-3,6,9,19,22-pentone; also known as Istodax, depsipeptide, FK228, FR901228, NSC630176) is a bicyclic depsipeptide originally isolated from Chromobacterium violaceum strain 968 (Ueda et al., 1994). Romidepsin is a histone deacetylase (HDAC) inhibitor that has been approved for the treatment of certain types of lymphoma. In both in vitro and in vivo systems, romidepsin has been shown to have pleiotropic activity that includes induction or repression of gene expression, cell cycle arrest, differentiation, cell growth inhibition, induction of apoptosis, morphological reversion of transformed cells, and inhibition of angiogenesis. Romidepsin exposure has been shown to modulate both the induction and repression of a number of key regulatory genes implicated in tumorigenesis, inflammation, autoimmune disorders, and immunomodulatory effects.

Most HDAC inhibitors are pan-HDAC inhibitors, implying they inhibit both Class I (HDACs 1, 2, 3, and 8) and Class II (HDACs 4, 5, 6, 7, 9, and 10) HDACs. In addition, they inhibit the sole Class IV HDAC referred to as HDAC 11. The clinically available HDAC inhibitors do not inhibit Class III HDACs (Sirtuins or Sirts). HDACs catalyze the removal of acetyl- groups from acetylated lysine residues in histones, resulting in changes in chromatin condensation and ultimately modulation of gene expression, which induces many of the cellular effects seen following exposure to inhibitors of these enzymes.

Of the various classes of HDAC enzymes, romidepsin most potently inhibits the Class I HDAC enzymes, which include HDACs 1, 2, 3 and 8. The changes in chromatin condensation seen following exposure to inhibitors of HDAC render DNA ‘transcriptionally active’ by maintaining an open chromatin structure known as euchromatin. In its condensed state, that facilitated by deacetylation of histone, chromatin is maintain in a transcriptionally repressed state. Romidepsin induces and represses the expression of numerous genes. Of more than 7000 genes examined in tumor cell lines using microarray analysis, approximately 100 were upregulated, while another 100 were downregulated following exposure to romidepsin. The pattern of altered gene expression varies, and can depend on many factors, including: (i) the cellular context; (ii) concentration of drug; (iii) duration of exposure to the drug; and (iv) concomitant medications. Consistently upregulated genes included p21WAF/Cip1, interleukin-8 (IL-8), and caspase 9, whereas consistently downregulated genes included mitogen-activated protein kinase (MAPK) and cyclin A2 (Sasakawa et al., 2005; Hoshino et al., 2007). Many of these genes encode proteins associated with critical regulatory functions in signal transduction, inhibition of growth, and apoptosis.

Importantly, in addition to inhibiting the deacetylation of HDAC, HDAC inhibitors can influence the acetylation status of non-histone proteins, influencing their post-translational state and subsequent function including but not limited to immunomodulatory effects (Ververis et al., 2013). The spectrum of these effects is less well understood, but includes important proteins involved in cancer biology, including Bcl-6 and p53.

Numerous studies have evaluated the in vitro and in vivo activity of romidepsin across multiple tumor cell lines. At nanomolar concentrations, romidepsin exhibited potent anticancer activity against both hematologic and solid tumor lines, including lymphoma, leukemia, and cancers of the prostate, kidney, colon, lung, stomach, breast, pancreas, as well as melanoma. Induction of anti-proliferative and pro-apoptotic activity in human B-cell chronic lymphocytic leukemia cells (CLL), B-cell prolymphocytic leukemia cells (PLL), T cell lymphoma cells, esophageal and pancreatic cancer cells, and multiple myeloma (MM) cells was seen at concentrations ranging from 1 to 500 nM. Romidepsin also showed potent cytotoxic effects on human lung, stomach, breast, and colon carcinoma cells, but exhibited weak cytotoxic effects on normal human cells.

The efficacy of romidepsin in vivo has been examined in numerous human xenograft studies in mice. In these studies, romidepsin has shown broad antitumor activity against multiple human tumor types, including those derived from epithelial, mesenchymal, and hematologic tissues (see e.g., Ueda et al., 1994). While the preclinical activity has not been a great predictor of clinical activity (that is, the drug is active in more preclinical models of cancer than has been demonstrated in the clinic), it is clear that romidepsin, and in fact this class of drugs regarded as HDAC inhibitors, have proven to have unique lineage selective activity across the T cell malignancies, and may complement certain immunologic treatments and other epigenetic drugs irrespective of its cell of origin.

### SUMMARY

This summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter related to compositions comprising, consisting essentially of, or consisting of a histone deacetylase inhibitor (HDACi) encapsulated in and/or otherwise associated with a nanoparticle. In some embodiments, the HDACi is selected from the group consisting of vorinostat, romidepsin, belinostat, panobinostat, and chidamide, or any combination thereof, optionally wherein the HDACi is romidepsin. In some embodiments, the nanoparticle is a poly(D,L-lactide)-PEG-methyl ether (mPEG-PDLLA) nanoparticle. In some embodiments, a composition of the presently disclosed subject matter comprises one or more polymers and/or one or more surfactants. In some embodiments, the one or more polymers are selected from the group consisting of a polyester, optionally PDLLA, PLGA, PLA, and/or PCL, copolymers thereof, and blends thereof. In some embodiments, the polymer comprises a polymer selected from the group consisting of a synthetic polymer; a biodegradable polymer; a biocompatible polymer; an amphiphilic polymer; a diblock co-polymer; and blends thereof. In some embodiments, the polymer comprises a hydrophilic, PEG chain, optionally methoxy PEG, PEG-carboxylic acid, PEG-hydroxyl, and/or PEG amine as end cap and chain length range 2K-10K. In some embodiments, the polymer is a hydrophobic core-forming polymer, optionally a hydrophobic core-forming polymer selected from the group consisting of PDLLA, PLGA, PLA, and/or PCL. In some embodiments, the nanoparticle comprises a methyl ether-PEG polylactide-co-glycolide (mPEG-PLGA, 50:50). In some embodiments, one or more parameters of the composition selected from a group consisting of mode of phase addition, HDACi/polymer ratio, HDACi/surfactant ratio, solvent/anti-solvent ratio, rate of addition, and combinations thereof are optimized. In some embodiments, the HDACi/polymer ratio ranges from about 1:10 to about 1:100 W/W, optionally 1:10 to about 1:50 W/W; the HDACi/surfactant ratio ranges from about 1:0.05 to about 1:0.2 W/W; the solvent/anti-solvent ratio ranges from about 1:10 to about 1:1, optionally wherein the anti-solvent is selected from the group consisting of water, PBS, or another ionic buffer solution; and/or the rate of addition ranges from about 10 to about 500 mL/hour, optionally about 10 to about 50 mL/hour.

The presently disclosed subject matter also relates in some embodiments to methods for treating diseases, disorders, and/or conditions associated with sensitivity to histone deacetylase inhibitors. In some embodiments, the methods comprise, consist essentially of, or consist of administering to a subject in need thereof an effective amount of a composition comprising, consisting essentially of, or consisting of a histone deacetylase inhibitor (HDACi) encapsulated in and/or otherwise associated with a nanoparticle. In some embodiments, the disease, disorder, and/or condition associated with sensitivity to histone deacetylase inhibitors is a tumor and/or a cancer. In some embodiments, the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

The presently disclosed subject matter also relates in some embodiments to methods for treating diseases, disorders, and/or conditions associated with sensitivity to a histone deacetylase inhibitor (HDACi) by administering to a subject in need thereof an effective amount of a composition as disclosed herein. In some embodiments, the disease, disorder, or condition associated with sensitivity to an HDACi is a tumor and/or a cancer, an inflammatory disease, disorder, or condition; an autoimmune disease, disorder, or condition; or any combination thereof. In some embodiments, the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

The presently disclosed subject matter also relates in some embodiments to methods for inhibiting the growth, proliferation, and/or metastasis of a tumor and/or a cancer associated with sensitivity to histone deacetylase inhibitors. In some embodiments, the methods comprise, consist essentially of, or consist of administering to a subject in need thereof an effective amount of a composition comprising, consisting essentially of, or consisting of a histone deacetylase inhibitor (HDACi) encapsulated in and/or otherwise associated with a nanoparticle. In some embodiments, the tumor and/or the cancer is selected from the group consisting of cutaneous T cell lymphoma (CTCL), peripheral T cell lymphoma (PTCL), and multiple myeloma. In some embodiments, the presently disclosed methods further comprise, consist essentially of, or consist of administering to the subject at least one additional therapeutically active agent. In some embodiments, the at least one additional therapeutically active agent is a chemotherapeutic agent. In some embodiments, the chemotherapeutic agent is selected from the group consisting of cyclophosphamide, doxorubicin; vincristine, prednisone, azacytidine, decitabine, cladribine, methotrexate, pralatrexate, and cyclosporin A, and combinations thereof.

The presently disclosed subject matter also relates in some embodiments to methods for inhibiting the growth, proliferation, and/or metastasis of a tumor and/or a cancer associated with sensitivity to a histone deacetylase inhibitor (HDACi). In some embodiments, the methods comprise, consist essentially of, or consist of administering to a subject in need thereof an effective amount of a composition as disclosed herein. In some embodiments, the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

In some embodiments, the presently disclosed methods further comprise, consist essentially of, or consist of administering to the subject at least one additional therapeutically active agent. In some embodiments, the at least one additional therapeutically active agent is a chemotherapeutic agent. In some embodiments, the chemotherapeutic agent is selected from the group consisting of cyclophosphamide, doxorubicin; vincristine, prednisone, azacytidine, decitabine, cladribine, methotrexate, pralatrexate, and cyclosporin A, and combinations thereof.

The presently disclosed subject matter also relates in some embodiments to methods for treating an inflammatory and/or an autoimmune disease, disorder, or condition. In some embodiments, the inflammatory and/or an autoimmune disease, disorder, or condition is selected from the group consisting of fatty liver disease, endometriosis, types 1 and 2 diabetes, inflammatory bowel disease, asthma, obesity, Alzheimer's and Parkinson's diseases, Ankylosing Spondylitis (AS), Antiphospholipid Antibody Syndrome (APS), Gout, Inflammatory Arthritis Center, Myositis, Rheumatoid Arthritis, Scleroderma, Sjogren's Syndrome, Systemic Lupus Erythematosus (SLE, Lupus), vasculitis, Addison's disease, Celiac disease-sprue (gluten-sensitive enteropathy), dermatomyositis, Grave's disease, Hashimoto's thyroiditis, Multiple sclerosis, Myasthenia gravis, Pernicious anemia, Reactive arthritis, Rheumatoid arthritis, Psoriasis/psoriatic arthritis, multiple sclerosis, Sjögren's syndrome, Systemic lupus erythematosus (SLE), type 1 diabetes, Inflammatory bowel disease (including Crohn's disease and ulcerative colitis), autoimmune vasculitis, Guillain-Barre syndrome, and Chronic inflammatory demyelinating polyneuropathy. In some embodiments, the presently disclosed methods comprise administering to a subject in need thereof a composition of the presently disclosed subject matter in combination with at least one additional therapeutically active agent. In some embodiments, the at least one additional therapeutically active agent is an anti-inflammatory and/or an immunosuppressant agent.

The presently disclosed subject matter also relates in some embodiments to methods for treating inflammatory and/or autoimmune diseases, disorders, and/or conditions associated with sensitivity to a histone deacetylase inhibitor (HDACi). In some embodiments, the method comprises, consists essentially of, or consists of administering to a subject in need thereof an effective amount of a composition as disclosed herein. In some embodiments, the inflammatory and/or an autoimmune disease, disorder, or condition is selected from the group consisting of fatty liver disease, endometriosis, types 1 and 2 diabetes, inflammatory bowel disease, asthma, obesity, Alzheimer's and Parkinson's diseases, Ankylosing Spondylitis (AS), Antiphospholipid Antibody Syndrome (APS), Gout, Inflammatory Arthritis Center, Myositis, Rheumatoid Arthritis, Scleroderma, Sjogren's Syndrome, Systemic Lupus Erythematosus (SLE, Lupus), vasculitis, Addison's disease, Celiac disease-sprue (gluten-sensitive enteropathy), dermatomyositis, Grave's disease, Hashimoto's thyroiditis, Multiple sclerosis, Myasthenia gravis, Pernicious anemia, Reactive arthritis, Psoriasis/psoriatic arthritis, multiple sclerosis, Systemic lupus erythematosus (SLE), type 1 diabetes, Inflammatory bowel disease (including Crohn's disease and ulcerative colitis), autoimmune vasculitis, Guillain-Barre syndrome, and Chronic inflammatory demyelinating polyneuropathy.

In some embodiments, the presently disclosed methods further comprise, consist essentially of, or consist of administering to the subject at least one additional therapeutically active agent. In some embodiments, the at least one additional therapeutically active agent is an anti-inflammatory and/or an immunosuppressant agent.

The presently disclosed subject matter also relates in some embodiments to methods for fabricating nanoparticles comprising one or more drug molecules. In some embodiments, the methods comprise, consist essentially of, or consist of: (a) varying in one or more iterations two or more parameters of a first or subsequent reaction mixture comprising a drug molecule and one or more polymers; (b) selecting a desired combination of parameters for a further reaction mixture based on the varying of step (a); and (c) precipitating a nanoparticle comprising the drug molecule from the further reaction mixture. In some embodiments, the reaction mixture further comprises a reaction mixture selected from the group consisting of a solvent, a non-solvent, a surfactant, and combinations thereof. In some embodiments, the solvent is an organic solvent. In some embodiments, the non-solvent is an aqueous solvent, water, or PBS buffer.

In some embodiments, the presently disclosed methods comprise optimization of one or more parameters selected from a group consisting of mode of phase addition, a drug/polymer ratio, a drug/surfactant ratio, solvent/anti-solvent ratio, rate of addition, and combinations thereof. In some embodiments, the drug is HDACi, optionally romidepsin. In some embodiments, the HDACi/polymer ratio ranges from about 1:10 to about 1:100 W/W, optionally 1:10 to about 1:50 W/W; the HDACi/surfactant ratio ranges from about 1:0.05 to about 1:0.2 W/W; the solvent/anti-solvent ratio ranges from about 1:10 to about 1:1, optionally wherein the anti-solvent is selected from the group consisting of water, PBS, or another ionic buffer solution; and/or the rate of addition ranges from about 10 to about 500 mL/hour, optionally about 10 to about 50 mL/hour.

Accordingly, it is an object of the presently disclosed subject matter to provide compositions and methods for treating diseases, disorders, and/or conditions associated with sensitivity histone deacetylase biological activities, including but not limited to tumors, cancers, inflammatory diseases, disorders, and/or conditions, and autoimmune diseases, disorders, and/or conditions.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and EXAMPLES, which are incorporated by reference and form part of the specification.

### BRIEF DESCRIPTIONS OF THE FIGURES

FIG. 1. Chemical structure of PDLLA polymer. Methoxy poly(ethylene glycol)-b-poly(DL-Lactide) mPEG-P(DL)LA; (Mw˜5,000:10,000 Da).

FIGS. 2A-2C. Light scattering data. Representative DLS graphs showing size distribution of particles containing romidepsin. FIG. 2A: neutral liposomes. FIG. 2B: neutral liposomes with 5% cholesterol. FIG. 2C: m-PEG PDLLA.

FIG. 3. Cryo-EM images of m-PEG NanoRomidepsin (NanoRomi) PDLLA (left panel) and Ghost mPEG-PDLLA (right panel) at 50 nm scale bar.

FIG. 4. Drug concentration of NanoRomidepsin formulations. Lane 1. Neutral liposomes; Lane 2. Neutral liposomes with 5% cholesterol; Lane 3. Polymer NanoRomidepsin particles at RT, and Lane 4. Polymer NanoRomidepsin particles at 4° C.

FIGS. 5A and 5B. Dose and Time dependent response of NanoRomidepsin Polymers in Cancer Cell Lines. FIG. 5A. Five different human cancer cell lines treated with a concentration range of romidepsin and NanoRomidepsin polymers with cell viability being assessed using the CELL TITER GLO® Assay at 60 hours. IC₅₀ values were determined based on cell line and time using GraphPad Prism software. FIG. 5B. IC₅₀ (nM) for romidepsin and NanoRomidepsin polymers for the 8 cell lines at each time point (30, 60, and 96 hours left to right bars, respectively, in each set of four data points in each panel of FIG. 5B).

FIGS. 6A and 6B. NanoRomidepsin Polymers induce Apoptosis in Cancer Cell lines. FIG. 6A. Representative flow cytometry dot plots showing analysis of cleaved poly-ADP-ribose polymerases (PARP) as an apoptotic marker after romidepsin and Nano-Romidepsin treatment of HH cell line at 30 hours. FIG. 6B. Graph showing an increase in the percentage of Cleaved PARP expressing HH cells, an indication of apoptosis, in a concentration dependent manner at 30 hours after treatment with romidepsin, NanoRomidepsin mPEG PDLLA PBS, NanoRomidepsin mPEG PDLLA H₂O, and NanoRomidepsin mPEG PLGA.

FIG. 7. NanoRomidepsin mPEG PDLLA is the most potent of the NanoRomidepsin polymers. Bar histograms comparing the potency of romidepsin and all three NanoRomidepsin polymers based on the IC₅₀ activity on all the cell lines tested. Each dot represents a cancer cell line.

FIGS. 8A and 8B. NanoRomidepsin PDLLA induces acetylation of histone proteins H3 and H4. (FIG. 8A) Representative Flow Cytometry Histograms (top panel) showing concentration dependent changes in the level of expression of AcH3 and AcH4 (i.e., acetylation [Ac] of Histone 3 [H3] and Histone 4 [H4]) in HH cells after treatment with romidepsin, and NanoRomidepsin mPEGPDLLA H₂O after 30 hours. Bar charts (bottom panel) showing the changes in the level of expression of AcH3 and AcH4 as measured by the Mean Fluorescence Intensity (MFI) in a concentration dependent manner in HH cells at 30 hours after treatment with romidepsin and NanoRomidepsin mPEG PDLLA H₂O (FIG. 8B) Western Blots analysis of HH cells for AcH3 and AcH4 following treatment with increasing concentrations of romidepsin and NanoRomidepsin PDLLA showing that the HH cell line exhibited increased levels of acetylation of H3 and H4 at 30 hours and with higher doses (3 nM and 30 nM) of both treatments.

FIG. 9. Dose and Time dependent response of romidepsin and NanoRomidepsin PDLLA Polymer in normal cells. PBMCs from 3 healthy donors were treated with a concentration range of romidepsin and NanoRomidepsin PDLLA and cell viability being assessed using the CELL TITER GLO® Assay at 24, 48, and 72 hours.

FIG. 10. In vivo efficacy, pharmacokinetics and pharmacodynamics studies using NanoRomidepsin PDLLA. Exemplary in vivo xenograft mouse model to compare the therapeutic response between NanoRomidepsin PDLLA and romidepsin.

FIGS. 11A-11D. Single dose toxicity study using NanoRomidepsin PDLLA. BALB/c mice were administered a single treatment of indicated doses of romidepsin and NanoRomidepsin PDLLA (NanoRomi). Body weight as a percentage of original for mice dosed with romidepsin (FIGS. 11A and 11C) and NanoRomi (FIGS. 11B and 11D). Depicted are percentage of body weight changes as percentage of starting weights with SEM (n=5 mice/group). FIGS. 11A and 11B: intraperitoneal administration. FIGS. 11C and 11D: intravenous administration.

FIGS. 12A-12D. Single dose toxicity study using NanoRomidepsin PDLLA. BALB/c mice were administered a single treatment of indicated doses of romidepsin and NanoRomi. Depicted are clinical scores after the treatment with romidepsin (FIGS. 12A and 12C) and NanoRomi (FIGS. 12B and 12D) with SEM (n=5 mice/group). FIGS. 12A and 12B: intraperitoneal administration. FIGS. 12C and 12D: intravenous administration.

FIGS. 13A and 13B. Pharmacokinetics study using NanoRomidepsin PDLLA. Plasma concentration-time dependence plot of romidepsin concentration in plasma after (FIG. 13A) intraperitoneal or (FIG. 13B) intravenous administration of a single treatment with free romidepsin (circles) or NanoRomidepsin PDLLA (squares).

FIGS. 14A and 14B. Repeated dose toxicity study using NanoRomidepsin PDLLA. Mice were administered a 2 mg/kg treatment of romidepsin or NanoRomidepsin PDLLA by intraperitoneal route of administration for arrow indicated days in BALB/c mice. Depicted are the percentage of body weight changes as a percentage of starting weights with SEM (FIG. 14A; n=5 mice/group) and clinical score with SEM (FIG. 14B; n=5 mice/group).

FIG. 15. Efficacy study using NanoRomidepsin PDLLA. Mice were administered a 2 mg/kg treatment of romidepsin or NanoRomidepsin PDLLA or equivalent volume vehicle (PBS) or ghost nanoparticle by intraperitoneal route of administration for arrow indicated days in H9-dtomato-luciferase tumor bearing mice. In vivo, BLI images were acquired to determine the response after the romidepsin and NanoRomidepsin PDLLA treatment. All cohorts were imaged on days 7, 10, 14, and 17 days (arrows) after post-engraftment using Lago X Spectrum Imaging System before the administration of drugs. After acquisition of images, a mean BLI curve of human H9 xenografts was generated where the y axis represents total flux (photon/s) and x axis represents time (Days) function.

FIGS. 16A and 16B. Exemplary nanoprecipitation method of the presently disclosed subject matter. FIG. 16A is an illustration of a traditional nanoprecipitation method. It is included to compare with the multi-pronged approach of the presently disclosed subject matter. FIG. 16B is a schematic representation of formulation and operational parameter optimization of nanoparticle properties in a parallel approach using a multi-channel syringe pump and multi-point stirrer. The scale-up of NanoRomidepsin formulation utilized the optimized parameters from FIG. 16B.

FIGS. 17A-17C: Light scattering and LC/MS data. Solvent Screening: Role of solvent on NanoRomdepsin NP preparation by the presently disclosed nanoprecipitation method. Depicted are bar graphs of the average diameter of particles (FIG. 17A), polydispersity (PDI; FIG. 17B), and romidepsin concentration (FIG. 17C) of NanoRomdepsin NPs prepared in tetrahydrofuran (THF), acetone plus 10% dimethylsulfoxide (DMSO), acetonitrile (ACN), and ethanol (EtOH).

FIGS. 18A-18D: Additional light scattering and LC/MS data. Di-block co-polymer optimization including, chain length of the polymer, ratio of m-PEG to block polymer, and molecular weight of the block polymer with respect to NPs properties in an exemplary nanoprecipitation method of the presently disclosed subject matter showing concentration effects of polymers. Depicted are bar graphs of average particle diameter (FIG. 18A), PDI of particles (FIG. 18B), drug concentration of NanoRomidepsin (FIG. 18C), and impact of drug:polymer ratio on drug concentration (FIG. 18D).

FIGS. 19A-19D: Additional light scattering and LC/MS data. Centrifugal Filter Optimization with respect to membrane cutoff on drug concentration, and removal of excess unencapsulated drug from the formulation visualized using a Rhodamine dye. Depicted are bar graphs of average particle diameter (FIG. 19A). Drug concentration of NanoRomidepsin (FIG. 19B), PDI of particles (FIG. 19C), and removal of free drug from the formulation visualized using Rhodamine dye (FIG. 19D).

FIGS. 20A-20D: Additional light scattering and LC/MS data. Optimization of effect of anti-solvent with respect to drug concentration. Depicted are bar graphs of average particle diameter (FIG. 20A). PDI of particles (FIG. 20B), drug concentration of NanoRomidepsin (FIG. 20C), and impact of organic to water ratio on drug concentration (FIG. 20D).

FIGS. 21A-21E: Additional light scattering and LC/MS data. Zeta potential and cryo-EM data and batch to batch reproducibility in size and concentration of the drug in multiple batches of scaled-up NanoRomidepsin formulation. Depicted are bar graphs of average particle diameter (FIG. 21A). PDI of particles (FIG. 21B), and drug concentration of NanoRomidepsin (FIG. 21C). FIGS. 21D-1 through 21D-3 show size distributions by intensity and zeta-potential distributions for Ghost particles (i.e., NPs without romidepsin; FIG. 21D-1), NanoRomidepsin NPs formed in water (FIG. 21D-2), and NanoRomidepsin NPs formed in 1×PBS (FIG. 21D-3). FIG. 21E is a cryo-electron micrograph of NanoRomidepsin particles formed as in FIGS. 21D-1 through 21D-3, respectively.

### I. GENERAL CONSIDERATIONS

### I.A. Clinical Approval of HDAC Inhibitors for the Treatment of Cancer.

Romidepsin (Istodax) was approved by the US Food and Drug Administration (FDA) in November 2009 for the treatment of patients with relapsed or refractory cutaneous T cell lymphoma (CTCL) who have received at least one prior systemic therapy. It received an expanded indication in May 2011 for the treatment of patients with relapsed or refractory (peripheral T cell lymphoma) PTCL in patients who have received at least one prior therapy. The indication in PTCL was granted under Accelerated approval (Table 1), while the approval in CTCL was a granted full approval. The indication in PTCL was recently withdrawn by the present sponsor of the drug secondary to a negative randomized phase 4 study.

The removal of romidepsin from the marketplace for patients with R/R PTCL has been seen as an unfortunate event, as many investigators who treat patients with these diseases lament the lack of effective therapies for patients with PTCL. As a single agent, romidepsin has produced a response in about 25% of patients. The important favorable feature of the drug has been its long duration of benefit, which can approximate well over a year in responding patients. This duration of benefit, also seen with other recently approved drugs for R/R PTCL, is considered a clinically meaning effect of the drug (see Table 1 for other agents approved in this disease). The negative randomized Phase 4 commitment study explored romidepsin in combination with a standard of care chemotherapy, called CHOP (i.e., Cyclophosphamide, Hydroxydaunorubicin hydrochloride (doxorubicin hydrochloride), ONCOVIN® (vincristine), and Prednisone). The regimen was found to produce excessive toxicity, limiting the amount of therapy any one patient could tolerate, likely leading to the negative study results. This is distinctly different from the merits of romidepsin when combined with rational combinations of other targeted drugs like the DNA methyltransferase (DNMT) inhibitors azacytidine and decitabine, and the antifol pralatrexate. In these combinations, romidepsin has exhibited potent synergy, which has translated to the clinic, where the two drug combinations have produced activity that has surpassed any other drug combination in the disease. Hence, the true value of romidepsin is likely to reside not necessarily in combination with chemotherapy, but with other rationally targeted drugs.

### IB. Toxicity of Romidepsin.

As of Nov. 4, 2020, approximately 11,985 patients with cancer have received romidepsin either as monotherapy or in combination (Celgene, 2021). The most common adverse reactions associated with romidepsin are gastrointestinal (nausea, vomiting, diarrhea, and constipation), hematologic (thrombocytopenia, leukopenia [neutropenia and lymphopenia], and anemia), and asthenic conditions (asthenia, fatigue, malaise, and lethargy). Serious and sometimes fatal infections, including pneumonia, sepsis, and viral reactivation including Epstein Barr and hepatitis B viruses, have been reported in clinical trials with romidepsin. Reactivation of Epstein Barr viral infection leading to liver failure has occurred in recipients of romidepsin. In fact, reactivation of EBV has resulted in a boxed warning on the Package Insert. Reactivation of hepatitis B virus (HBV) infection has occurred in 1.1% of PTCL patients in clinical trials in United States (US), Australia and Europe. Other types of events commonly seen with romidepsin may include electrolyte abnormalities (hypomagnesemia, hypokalemia, hypocalcemia), pyrexia, and taste disturbances. There have also been few reports of hypersensitivity reactions with romidepsin (Kakar et al., 2020).

The most serious adverse event associated with romidepsin has been cardiotoxicity. Prolongation of QTc as well as several changes in electrocardiograms (ECG) (including T-wave and ST segment changes) have been reported in clinical studies. The initial clinical experiences with romidepsin resulted in several Grade 5 deaths due to cardiac arrhythmias, which were attributed to drug: drug interactions, namely combinations that included particular antiemetic agents. These ECG changes were transient and were not associated with functional cardiovascular changes or with symptoms, though there is a well-documented fatal cardiac arrhythmia associated with use of the drug.

Presently, romidepsin continues to carry an approval for patients with relapsed or refractory CTCL. As noted above, the data from a recently reported randomized Phase 3 (part of the Phase 4 commitment) of romidepsin plus CHOP based chemotherapy (referred to as the Ro-CHOP study; Romidepsin, Cyclophosphamide, Hydroxydaunorubicin hydrochloride (doxorubicin hydrochloride), ONCOVIN® (vincristine), and Prednisone; NCT01796002) conducted in adult patients with previously untreated PTCL was reported as negative as it did not meet the primary endpoint in demonstrating an improvement in progression free survival (PFS; Bachy et al., 2020). The toxicity profile of Ro-CHOP was substantial and found to be consistent with its phase Ib/II data study this particular combination, with no unexpected findings. The high rates of treatment emergent adverse events (TEAEs) with the addition of romidepsin hampered the ability to adequately administer 6 cycles of CHOP. Thus the combination of CHOP plus romidepsin does not represent an advance in the standard of care for patients with previously untreated PTCL (Bachy et al., 2020). These collective factors, the recent withdrawal from the market, the excessive toxicity seen in combination with chemotherapy, coupled to the fact that romidepsin does address an important unmet medical need for patients with PTCL, creates an unprecedented opportunity to develop versions of the drug that will remediate its liabilities while augmenting its efficacy.

### I.C. Advantages of Nano Polymer Romidepsin

Nano medicine offers nanoscale “solutions” for small molecule therapeutics to improve pharmacokinetics, bioavailability, and toxicological profiles, as well as targeted delivery. In addition, our group has been on the cutting edge of developing Nano formulations of oncological, neurological, and metabolic drugs to increase their therapeutic indices and extend IP protection. To improve physio-chemical properties and associated drug toxicities of romidepsin, optimization of a nanotechnology derived version of the drug is considered a highly promising approach to deliver romidepsin because it allows loading and release of this drug in an efficient, specific, and controlled manner. The unique properties of nanoparticles, such as their small size, large surface-to-volume ratios, the ability to create combinatorial nano-therapeutics, and the ability to achieve multivalency of targeting ligands on their surface, provide superior advantages for nanoparticle-based drug delivery for a variety of cancers. In addition, it has been widely observed that nano-therapeutics have a substantially greater penchant for the tumor microenvironment, which has the benefit of reducing or completely eliminating off target toxicities. Based on these principles, it is suspected that a nano polymer of romidepsin will be able to resolve many of the challenges of romidepsin, likely producing a superior safety profile, markedly improved scheduling and possibly superior activity and efficacy. We anticipate that the improved safety profile will substantially broaden the opportunities to combine romidepsin with other effective therapeutics.

Our group has pioneered novel therapeutic combinations with romidepsin including novel Nano-therapeutic polymers. Based on emerging data demonstrating, in preclinical and clinical experiences, the profound synergy seen with romidepsin plus other agents active in T cell malignancies, we have developed a platform to configure novel and original ratiometric nano polymer drugs predicated on romidepsin. This strategy, now being applied across the panoply of drugs active in PTCL, offers the prospect of creating a new standard of care for the disease, and to change the natural history of this challenging disease.

### II. DEFINITIONS

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4).

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

“Amphiphilic polymers” as used herein, describe polymer materials comprising both hydrophilic and hydrophobic unit chains. In some embodiments, various polymers control NP properties, polymers include but are not limited to m-PEG-PLGA, m-PEG-PCL and m-PEG-PDLLA of various respective chain lengths of hydrophobic core and PEG, which can confer a “stealth” property.

The term “aqueous solution” as used herein can include other ingredients commonly used, such as sodium bicarbonate described herein, and further includes any acid or base solution used to adjust the pH of the aqueous solution while solubilizing a peptide.

“Batch-to-batch” as used herein, describes the manner by which the formulation is reproducible, with optimal variation between batches in the context of physio-chemical properties especially for drug loading.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

“Biodegradable” as used herein, refers to the materials that can break down inside the body to non-toxic natural products and can be easily eliminated. In general, biocompatible and biodegradable are often associated with PLA and PLGA polymers comprising ester bonds. In some embodiments, the breakdown of these polymers are due to cellular or in vivo biological actions not by hydrolysis. The polymers with biodegradable properties play a role in the drug release. In some embodiments, drug release is governed by cleavage of polymer bonds, erosion of polymer matrix, and diffusion of encapsulated drug from the particles.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

“Centrifugal filters” as used herein, describe the materials used to process nanoparticles to remove excess unencapsulated drug by centrifugation or ultracentrifugation methods. In some embodiments, a centrifugal filter that is employed in the methods of the presently disclosed subject matter has a cutoff range of about 3K to about 100K.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein with reference to drugs and drug interactions refers to an interaction or interactions among one or more therapeutic agents that results in a greater benefit to a subject than would have occurred if the one or more therapeutic agents were not given to the subject. In some embodiments, a complementary drug interaction results in a synergistic benefit to the subject.

“Complementary” as used herein with reference to biomolecules refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

“Combinatorial” and “combinatorial fashion” as used herein, describes the process of utilizing multiple parameters in a single experiment to compare the physiochemical properties of nanoparticles.

“Concentration”, as used herein, is a measurement of the quantity of drug in the nanoparticles. In general, concentration of drug in nanoparticles is impacted by both mechanical process and formulation process, including solvent and anti-solvent ratio, drug to polymer ratio, mixing speed, solvent properties including density, D, dielectric constant, polarity, viscosity, eluent strength, etc. In some embodiments, particles can contain romidepsin in a concentration including, but not limited to 20 μg/ml to 1000 μg/mL.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is leukemia, which in some embodiments is Acute Myeloid Leukemia (AML).

“Controlled addition” as used herein, describes the addition of one phase to another phase by fixed rate of addition (in some embodiments, from about 10 to about 500 mL/hour) by using a syringe pump to produce particles with reproducible properties including but not limited to size, PDI, zeta potential, and drug loading.

“Cryo-protectant”, as used herein, describes the excipient or a stabilizer to protect the stability of NPs during the lyophilization process, including but not limited to an excipient such as mannitol, glucose, and the like.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encapsulation efficiency” as used herein, designates % of drug encapsulated with in the particle compared to the total drug used.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

“Formulation variables” as used herein describe a variable or parament to be considered for modification in a reaction mixture for a nanoparticle, including but not limited to drug to polymer ratio, choice of surfactant, surfactant addition to solvent or nonsolvent, water to organic ratio, a parameter that contributes to improvement of encapsulation efficiency.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Hydrophilic” as used herein, refers to substances containing high polar groups that readily interact with and/or are soluble in water.

“Hydrophobic” as used herein, refers to substances containing less polar groups and are typically characterized by low solubility in water.

As used herein, the phrases “inhibitor of HDAC activity”, “HDAC inhibitors (HDACi)”, and grammatical variants thereof refer to inhibitors of at least one biological activity of at least one histone deacetylase (HDAC), whether in vitro, in vivo, or ex vivo. Exemplary HDAC inhibitors include, but are not limited to romidepsin, vorinostat, panobinostat, belinostat, and chidamide (i.e., N-(2-Amino-4-fluorophenyl)-4-[[[(E)-3-pyridin-3-ylprop-2-enoyl]amino]methyl]benzamide). Other compounds that can serve as HDAC inhibitors include valproic acid, trichostatin A, butyric acid and its derivatives including but not limited to 4-phenylbutyric acid, entinostat, givinostat, droxinostat, tubastatin A, pracinostat, and others.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

“Lyophilization” as used herein, describes the process utilized to create powder form of NPs with or without cryo-protectants to improve the shelf-life, dosage logistics, and storage logistics.

“Mean particle size” used herein, generally refers in some embodiments to spherical particles' hydrodynamic diameter. In general, NP size plays a role in macrophage uptake over a surface chemistry, such as a PEG surface chemistry. In some embodiments, a lower molecular weight of a polymer used to prepare a NP contributes to smaller size NPs resulting in altered drug release kinetics, higher circulation, less accumulation in organs like liver and spleen, larger exposure of drug contributes to enhanced biological activity.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The phrase “molecular weight”, as used herein, refers to the chain length of bulk polymer. In some embodiments, physical properties like solubility, viscosity, crystallinity, mechanical strength, and degradation rate can depend on the molecular weight of the polymer.

“Monodisperse” and homogenous solutions are used as synonyms or interchangeably, used herein, particles of same or almost same diameter distribution is referred as monodisperse.

“Multi-variant” as used herein, describes the number of variables included into the preparation of NPs that determines the physio-chemical properties of the drug.

The terms “nano”, “nanomaterial”, “nanoparticle”, and “NP” as used herein, refer to a structure having at least one region with a dimension and/or size (e.g., length, width, diameter, etc.) less than or equal to about 1,000 nm including all integers or fractional integers in between (such as but not limited to 30, 40, 50, 60, 70, 80, 90, 100, 200, 500, 1,000 nm). In some embodiments, the dimension is smaller (e.g., less than about 500 nm, less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm). In some embodiments, the dimension is between about 20 nm and about 250 nm (e.g., about 20, 30, 40, 50, 60, 70, 80, 90, 100, 110, 120, 130, 140, 150, 160, 170, 180, 190, 200, 210, 220, 230, 240, or 250 nm). In general, particles occupy size ranges between that which induce size-dependent rapid renal clearance and prevent size-dependent liver accumulation. The present disclosed compositions might exist in a variety of shapes, including, but not limited to core-shell, circular, spherical, spheroidal, and micellar. In general, nanoparticles having a spherical shape are referred to as Nano spheres.

In some embodiments, nanoparticles, including “core-shell” nanoparticles, are formed from biocompatible, biodegradable, block co-polymers in amphiphilic nature. In some embodiments, a core-shell nanoparticle is a nanoparticle made out of di-block polymers wherein the shell is hydrophilic and the core is hydrophobic.

“Nanoprecipitation” as used herein refers in some embodiments to the method of making polymer NPs by bottom-up approach. In some embodiments of this method, one phase is added to another phase under moderate magnetic stirring. In some embodiments, nanoprecipitation is employed to encapsulate hydrophobic drugs. It is chosen because of its simplicity, scalability, and batch-to-batch reproducibility by controlled addition. This method facilitates multi-parameter optimization in a combinatorial fashion to achieve the desired properties such as size, zeta potential, and drug loading.

“Operating variables” as used herein, describe a mechanical variable or parament to be considered for modification including, but not limited to mechanical speed, mixing solvents, mode of addition, rate of addition, centrifugation speed, and time to improve the physiochemical properties of colloidal solution.

“Optimization” as used herein, describes the process of finding a desirably effective drug concentration in particles to exert therapeutic effect. In some embodiments, a multi-pronged approach is used to optimize drug concentration by including process and formulation parameters in a combinatorial fashion. “Multi-pronged” as used herein, describes the process of approach to engineering the nanoprecipitation method to optimize the physiochemical properties of Nanoparticles. In general, nanoprecipitation method facilitates parameter optimization. In other embodiments, a syringe pump with a multi-channel syringe system is applied to engineer nanoparticles.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

“Parallel” as used herein refers to an approach for nanoparticle fabrication involving multi-parameter optimization in a combinatorial fashion and it is often used as synonym of combinatorial approach.

“Parameter” as used herein, describes a parameter to be considered for modification with respect to physiochemical properties of NPs. In general, there are process and formulation related parameters which impact size, charge, and drug encapsulation efficiency.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intratumoral, and kidney dialytic infusion techniques.

In some embodiments, “particle count” or “population” refer to a group of particles including nanoparticles, including particles with uniform size, charge, shape, and composition, including nanoparticles with uniform size, charge, shape, and composition.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

As used herein, the term “mammal” refers to any member of the class Mammalia, including, without limitation, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Scalability” as used herein, describes the process of taking the nanoformulations from small scale to bulk to conduct in vitro and in vivo experiments to test the PK and PD effect of formulation.

As used herein, the phrase “sensitivity to histone deacetylase inhibitors” refers to a cell, tissue, or organ in which one or more undesirable histone deacetylase biological activities occur and/or have an effect that can be improved by treatment with a histone deacetylase inhibitor. In some embodiments, the undesirable histone deacetylase biological activity is associated with a disease, disorder, or condition that at least one symptom of which is improved and/or inhibited by treatment with a histone deacetylase inhibitor either alone or in combination with other treatments.

“Solvent” as used herein, refers in some embodiments to an organic substance used and, in some embodiments, acts as solvent for film-forming materials, romidepsin, and polymer. In some embodiments polar solvents, including water-miscible solvents, ethanol, dimethylsulfoxide (DMSO), acetone, tetrahydrofuran (THF), acetonitrile, or a combination of solvents are used to dissolve drug and polymer for preparation of nanoparticles. The physical properties of solvent influence the solubility of drug and polymer including the physio-chemical properties of NPs and overall nanoprecipitation method of making NPs. Acetonitrile is a particular example of solvent used to dissolve drug, polymer, and surfactant to prepare NPs by in a nanoprecipitation method. In some embodiments, acetonitrile (ACN) provided the greatest concentration, lowest PDI, and an average size. In addition to ACN, other solvents would be apparent to one of ordinary skill in the art upon a review of the instant disclosure.

“Anti-solvent” as used herein, refers an aqueous solvent, including but not limited to water or a buffer solution such as PBS, that can be employed to disperse solvent containing drug and polymer. Non-solvent and anti-solvent terms are interchangeable. In some embodiments, hydrophilic excipients can also be added to a non-solvent.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

“Surfactant” is used herein as a material that supports NPs stability. In some embodiments, a surfactant is a compound that decreases the surface tension between two liquids, between a gas and a liquid, or between a liquid and a solid. In some embodiments, a surfactant composition includes but is not limited to a polaxamer (e.g., polaxamer 188, polaxamer 237, polaxamer 338, and polaxamer 407), Tween 20, Tween 80, polyvinyl alcohol (PVA), etc. In general, these materials are used as emulsifiers and can avoid aggregation.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

As used herein, the phrase “zeta potential” refers to a measurement of surface potential of a particle. In some embodiments of the presently disclosed subject matter, the particles have a zeta potential in the range of −25 to +25.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### III. EXEMPLARY EMBODIMENTS

In some embodiments, the presently disclosed subject matter relates to the compositions comprising HDACi polymer nanoparticles, the parallel approach used making nanoparticles, and the multivariable parameters included in optimizing drug concentration within the nanoparticle. The method herein discloses the modified parameters/operating conditions, including but not limited to: drug/polymer ratio, solvent/water ratios, mixing speed, controlled addition and processing methods used in producing HDACi NPs. In other embodiments, the hydrophobic m-PEG PDLLA (5K:10K) polymer was used in encapsulating HDACi to improve the PK profile of the drugs. In some embodiments, the compositions of smaller HDACi NPs comprises amorphous m-PEG PDLLA polymer leading to improved drug release kinetics. In some embodiments, the HDACi is encapsulated in and/or otherwise associated with a nanoparticle. In some embodiments, the combination of the representative parameters were optimized with respect to drug loading and was chosen to “lock down” the formulation. In some embodiments, the lockdown formulation parameters facilitated the scale-up of HDACi NPs for in vivo studies. In some embodiments, the optimized high throughput parallel synthesis or combinatorial approach allowed us to achieve volumes of 300-400 mL of nanoformulation at the current concentration, about 500 μg/mL, in a very cost and energy-efficient manner. An exemplary nanoparticle is a poly(D,L-lactide)-PEG-methyl ether (mPEG-PDLLA) nanoparticle. Some nanoparticles may employ or include other polymers, surfactants, lipids, or a combination of polymer and lipid. They may also employ other methods to produce core-shell, circular, sphere-shaped, spherical, micellar, mono, and bilayer polymersomes to compose one or more histone HDACis.

Thus, in some embodiments, the presently disclosed subject matter relates in some embodiments to compositions for use in preventing and/or treating a disease, disorder, and/or condition associated with sensitivity to HDAC inhibitors. In some embodiments, the presently disclosed subject matter relates to compositions comprising, consisting essentially of, or consists of one or more histone deacetylase inhibitors (HDACi). Exemplary HDACi include vorinostat, romidepsin, belinostat, panobinostat, and chidamide.

In some embodiments, the HDACi is encapsulated in and/or otherwise associated with a nanoparticle. An exemplary nanoparticle is a poly(D,L-lactide)-PEG-methyl ether (mPEG-PDLLA) nanoparticle, although nanoparticles may employ or include other lipids, organic molecules, and/or inorganic molecules.

As such, in some embodiments the presently disclosed subject matter related to compositions comprising, consisting essentially of, or consisting of a histone deacetylase inhibitor (HDACi) encapsulated in and/or otherwise associated with a nanoparticle. In some embodiments, the HDACi is selected from the group consisting of vorinostat, romidepsin, belinostat, panobinostat, and chidamide, or any combination thereof, optionally wherein the HDACi is romidepsin. In some embodiments, the nanoparticle is a poly(D,L-lactide)-PEG-methyl ether (mPEG-PDLLA) nanoparticle. In some embodiments, a composition of the presently disclosed subject matter comprises one or more polymers and/or one or more surfactants. In some embodiments, the one or more polymers are selected from the group consisting of a polyester, optionally PDLLA, PLGA, PLA, and/or PCL, copolymers thereof, and blends thereof. In some embodiments, the polymer comprises a polymer selected from the group consisting of a synthetic polymer; a biodegradable polymer; a biocompatible polymer; an amphiphilic polymer; a diblock co-polymer; and blends thereof. In some embodiments, the polymer comprises a hydrophilic, PEG chain, optionally methoxy PEG, PEG-carboxylic acid, PEG-hydroxyl, and/or PEG amine as end cap and chain length range 2K-10K. In some embodiments, the polymer is a hydrophobic core-forming polymer, optionally a hydrophobic core-forming polymer selected from the group consisting of PDLLA, PLGA, PLA, and/or PCL. In some embodiments, the nanoparticle comprises a methyl ether-PEG polylactide-co-glycolide (mPEG-PLGA, 50:50). In some embodiments, one or more parameters of the composition selected from a group consisting of mode of phase addition, HDACi/polymer ratio, HDACi/surfactant ratio, solvent/anti-solvent ratio, rate of addition, and combinations thereof are optimized. In some embodiments, the HDACi/polymer ratio ranges from about 1:10 to about 1:100 W/W, optionally 1:10 to about 1:50 W/W; the HDACi/surfactant ratio ranges from about 1:0.05 to about 1:0.2 W/W; the solvent/anti-solvent ratio ranges from about 1:10 to about 1:1, optionally wherein the anti-solvent is selected from the group consisting of water, PBS, or another ionic buffer solution; and/or the rate of addition ranges from about 10 to about 500 mL/hour, optionally about 10 to about 50 mL/hour.

### III.A. Formulations

The compositions (e.g., HDAC NPs) of the presently disclosed subject matter can be administered in any formulation or route that would be expected to deliver the compositions to whatever target site might be appropriate.

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

### III.B. Administration

Suitable methods for administration of the compositions of the presently disclosed subject matter include, but are not limited to intravenous administration and delivery directly to the target tissue or organ (e.g., a tumor, a cancer, or endothelial tissue associated therewith). Exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated.

### III.C. Doses

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated, such as but not limited to a reduction in the growth and/or proliferation of a tumor and/or a cancer, and/or a reduction in the extent to and/or timing at which a disease, disorder, and/or condition develops in a subject). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore a “treatment effective amount” can vary. However, using the methods described herein, one skilled in the art can readily assess the potency and efficacy of a composition of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease, disorder, and/or condition treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

In some embodiments, the presently disclosed subject matter also relates to methods for treating a disease, disorder, or condition associated with sensitivity to histone deacetylase inhibitors, the method comprising administering to a subject in need thereof an effective amount of a composition as disclosed herein. In some embodiments, the disease, disorder, or condition associated with sensitivity to histone deacetylase inhibitors is a tumor and/or a cancer. In some embodiments, the tumor and/or the cancer is selected from the group consisting of cutaneous T cell lymphoma (CTCL), peripheral T cell lymphoma (PTCL), and multiple myeloma. In some embodiments, the disease, disorder, or condition associated with sensitivity to histone deacetylase inhibitors is an autoimmune disease, disorder, or condition, which in some embodiments can be large granular lymphocytic leukemia.

As such, the presently disclosed subject matter also relates in some embodiments to methods for treating diseases, disorders, and/or conditions associated with sensitivity to histone deacetylase inhibitors. In some embodiments, the methods comprise, consist essentially of, or consist of administering to a subject in need thereof an effective amount of a composition comprising, consisting essentially of, or consisting of a histone deacetylase inhibitor (HDACi) encapsulated in and/or otherwise associated with a nanoparticle. In some embodiments, the disease, disorder, and/or condition associated with sensitivity to histone deacetylase inhibitors is a tumor and/or a cancer. In some embodiments, the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

The presently disclosed subject matter also relates in some embodiments to methods for treating diseases, disorders, and/or conditions associated with sensitivity to a histone deacetylase inhibitor (HDACi) by administering to a subject in need thereof an effective amount of a composition as disclosed herein. In some embodiments, the disease, disorder, or condition associated with sensitivity to an HDACi is a tumor and/or a cancer, an inflammatory disease, disorder, or condition; an autoimmune disease, disorder, or condition; or any combination thereof. In some embodiments, the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

In some embodiments, the presently disclosed subject matter also relates to methods for inhibiting the growth, proliferation, and/or metastasis of a tumor and/or a cancer associated with sensitivity to histone deacetylase inhibitors. In some embodiments, the methods comprise, consist essentially of, or consist of administering to a subject in need thereof an effective amount of a composition as disclosed herein. In some embodiments, the tumor and/or the cancer is selected from the group consisting of cutaneous T cell lymphoma (CTCL), peripheral T cell lymphoma (PTCL), and multiple myeloma.

As such, the presently disclosed subject matter also relates in some embodiments to methods for inhibiting the growth, proliferation, and/or metastasis of a tumor and/or a cancer associated with sensitivity to histone deacetylase inhibitors. In some embodiments, the methods comprise, consist essentially of, or consist of administering to a subject in need thereof an effective amount of a composition comprising, consisting essentially of, or consisting of a histone deacetylase inhibitor (HDACi) encapsulated in and/or otherwise associated with a nanoparticle. In some embodiments, the tumor and/or the cancer is selected from the group consisting of cutaneous T cell lymphoma (CTCL), peripheral T cell lymphoma (PTCL), and multiple myeloma. In some embodiments, the presently disclosed methods further comprise, consist essentially of, or consist of administering to the subject at least one additional therapeutically active agent. In some embodiments, the at least one additional therapeutically active agent is a chemotherapeutic agent. In some embodiments, the chemotherapeutic agent is selected from the group consisting of cyclophosphamide, doxorubicin; vincristine, prednisone, azacytidine, decitabine, cladribine, methotrexate, pralatrexate, and cyclosporin A, and combinations thereof.

The presently disclosed subject matter also relates in some embodiments to methods for inhibiting the growth, proliferation, and/or metastasis of a tumor and/or a cancer associated with sensitivity to a histone deacetylase inhibitor (HDACi). In some embodiments, the methods comprise, consist essentially of, or consist of administering to a subject in need thereof an effective amount of a composition as disclosed herein. In some embodiments, the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

In some embodiments, the presently disclosed methods further comprise, consist essentially of, or consist of administering to the subject at least one additional therapeutically active agent. In some embodiments, the at least one additional therapeutically active agent is a chemotherapeutic agent. In some embodiments, the chemotherapeutic agent is selected from the group consisting of cyclophosphamide, doxorubicin; vincristine, prednisone, azacytidine, decitabine, cladribine, methotrexate, pralatrexate, and cyclosporin A, and combinations thereof.

### III.D. Combination Therapies

In some embodiments, the presently disclosed subject matter relates to combination therapies in which a given disease, disorder, or condition associated with sensitivity to histone deacetylase inhibitors is treated with an HDAC inhibitor and also one or more additional therapeutic agents that are appropriate for the disease, disorder, or condition to be treated. Thus, in some embodiments, the presently disclosed methods can further comprise, consist essentially of, or consist of administering to the subject at least one additional therapeutically active agent. In some embodiments wherein the disease, disorder, or condition to be treated is a tumor and/or a cancer, the at least one additional therapeutically active agent can be a chemotherapeutic agent. Chemotherapeutic (cytotoxic) agents including, but are not limited to, 5-fluorouracil, bleomycin, busulfan, camptothecins, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raioxifene, tamoxifen, taxol, temazolomide (an aqueous form of DTIC), transplatinum, vinblastine, methotrexate, vincristine, and any analogs and/or derivatives or variants of the foregoing. Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog, derivative, or variant thereof. In some embodiments, the chemotherapeutic agent is selected from the group consisting of cyclophosphamide, doxorubicin; vincristine, and prednisone, and combinations thereof. In some embodiments, the at least one additional therapeutically active agent is cyclosporine A (CSA), a hypomethylating agent, cladribine, or pralatrexate. In some embodiments, the at least one additional therapeutically active agent is an active agent that targets PI3K, Bcl-2, BTK, HDAC, DNMT, BRAF, or MEK, and/or is an active agent that is classified as targeting epigenetic phenomena, and/or is an immunologic therapeutic such as but not limited to a monoclonal antibody, an antibody/drug conjugate, a bispecific antibody, and/or an adoptive cellular therapy such as but not limited to a CAR-T cell or CAR-T-based therapeutic including but not limited to commercially available T cell therapeutics.

In some embodiments, the tumor and/or the cancer is sensitive to and/or refractory, relapsed, and/or resistant to one or more chemotherapeutic agents such as, but not limited to a platinum-based agent, a taxane, an alkylating agent, an anthracycline (e.g., doxorubicin including but not limited to liposomal doxorubicin), an antimetabolite, and/or a vinca alkaloid. In some embodiments, the cancer is an ovarian cancer, and the ovarian cancer is refractory, relapsed, or resistant to a platinum-based agent (e.g., carboplatin, cisplatin, oxaliplatin), a taxane (e.g., paclitaxel, docetaxel, larotaxel, cabazitaxel), and/or an anthracycline (e.g., doxorubicin including but not limited to liposomal doxorubicin). In some embodiments, the cancer is colorectal cancer, and the cancer is refractory, relapsed, or resistant to an antimetabolite (e.g., an antifolate (e.g., pemetrexed, floxuridine, raltitrexed) a pyrimidine analogue (e.g., capecitabine, cytrarabine, gemcitabine, 5FU)), and/or a platinum-based agent (e.g., carboplatin, cisplatin, oxaliplatin). In some embodiments, the cancer is lung cancer, and the cancer is refractory, relapsed, or resistant to a taxane (e.g., paclitaxel, docetaxel, larotaxel, cabazitaxel), a platinum-based agent (e.g., carboplatin, cisplatin, oxaliplatin), a vinca alkaloid (e.g., vinblastine, vincristine, vindesine, vinorelbine), a vascular endothelial growth factor (VEGF) pathway inhibitor, an epidermal growth factor (EGF) pathway inhibitor) and/or an antimetabolite (e.g., an antifolate including but not limited to pemetrexed, floxuridine, or raltitrexed), and a pyrimidine analogue (e.g., capecitabine, cytrarabine, gemcitabine, 5FU). In some embodiments, the cancer is breast cancer, and the cancer is refractory, relapsed, or resistant to a taxane (e.g., paclitaxel, docetaxel, larotaxel, cabazitaxel), a VEGF pathway inhibitor, an anthracycline (e.g., daunorubicin, doxorubicin including but not limited to liposomal doxorubicin, epirubicin, valrubicin, idarubicin), a platinum-based agent (e.g., carboplatin, cisplatin, oxaliplatin), and/or an antimetabolite (e.g., an antifolate including but not limited to pemetrexed, floxuridine, or raltitrexed), and a pyrimidine analogue (e.g., capecitabine, cytrarabine, gemcitabine, 5FU). In some embodiments, the cancer is gastric cancer, and the cancer is refractory, relapsed, or resistant to an antimetabolite (e.g., an antifolate including but not limited to pemetrexed, floxuridine, raltitrexed) and a pyrimidine analogue (e.g., capecitabine, cytrarabine, gemcitabine, 5FU) and/or a platinum-based agent (e.g., carboplatin, cisplatin, oxaliplatin). In some embodiments, the provision of the HDACi as part of a nanoparticle overcomes the tumor's and/or the cancer's nature as being refractory, relapsed, and/or resistant to one or more chemotherapeutic agents. Alternatively or in addition, the compositions and methods of the presently disclosed subject matter can include active agents that are routinely used in the treatment of, for example, lymphoma including but not limited to alkylating agents (e.g., cyclophosphamide, ifosphamide dacarbazine, and BCNU), anthracyclines, vinca alkaloids, platinum analogs, antimetabolites (e.g., methotrexate, Ara-C, gemcitabine), topoisomerase inhibitors, steroids, and combinations thereof.

In some embodiments wherein the disease, disorder, or condition to be treated is an inflammatory disease, disorder, or condition. Exemplary, non-limiting inflammatory diseases, disorders, or conditions include Fatty liver disease, endometriosis, types 1 and 2 diabetes, inflammatory bowel disease, asthma, obesity, Alzheimer's and Parkinson's diseases, Ankylosing Spondylitis (AS), Antiphospholipid Antibody Syndrome (APS), Gout, Inflammatory Arthritis Center, Myositis, Rheumatoid Arthritis, Scleroderma, Sjogren's Syndrome, Systemic Lupus Erythematosus (SLE, Lupus), Vasculitis, and tumors/cancers. In some embodiments, the at least one additional therapeutically active agent can thus be any anti-inflammatory agent typically employed in the treatment/management of any of these diseases, disorders, or conditions. Exemplary anti-inflammatory agents include, but are not limited to Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium; methotrexate, dexamethasone, dexamethasone alcohol, dexamethasone sodium phosphate, fluromethalone acetate, fluromethalone alcohol, lotoprendol etabonate, medrysone, prednisolone acetate, prednisolone sodium phosphate, difluprednate, rimexolone, hydrocortisone, hydrocortisone acetate, lodoxamide tromethamine, glucocorticoids, diclofenac, and any combination thereof.

In some embodiments wherein the disease, disorder, or condition to be treated is an autoimmune disease, disorder, or condition. Exemplary autoimmune diseases, disorders, or conditions include Addison's disease, Celiac disease-sprue (gluten-sensitive enteropathy), dermatomyositis, Grave's disease, Hashimoto's thyroiditis, Multiple sclerosis, Myasthenia gravis, Pernicious anemia, Reactive arthritis, Rheumatoid arthritis, Psoriasis/psoriatic arthritis, multiple sclerosis, Sjögren's syndrome, Systemic lupus erythematosus (SLE), type 1 diabetes, Inflammatory bowel disease (including Crohn's disease and ulcerative colitis), autoimmune vasculitis, Guillain-Barre syndrome, and Chronic inflammatory demyelinating polyneuropathy. In some embodiments, the at least one additional therapeutically active agent can thus be any therapeutic agent typically employed in the treatment/management of any of these diseases, disorders, or conditions, including but not limited to the anti-inflammatories listed above and/or steroids (including but not limited to prednisone, methylprednisolone, and dexamethasone), colchicine, hydroxychloroquine (Plaquenil), Sulfasalazine, dapsone, methotrexate, Mycophenolate Mofetil (Cellcept, Myfortic), Azathioprine (Imuran), anti-IL-1 biologics including but not limited to Anakinra/Kineret, Canakinumab/Ilaris, and Rilonacept/Arcalyst), anti-TNF biologics (including but not limited to nfliximab/Remicade, Adalimumab/Humira, Golimumab/Simponi, Etanercept/Enbrel, and Certolizumab/Cimzia), anti-IL-6 biologics (including but not limited to Tocilizumab/Actemra and Sarilumab/Kevzara), complement inhibitors (including but not limited to Eculizumab), anti-CD20 biologics (including but not limited to Rituximab/Rituxan), B cell growth factor targeting biologics (including but not limited to Belimumab/Benlysta), therapeutics targeted to adaptive immunity-T cells (including but not limited to cyclosporine), therapeutics targeted to T cell co-stimulation and/or activation (including but not limited to Abatacept/Orencia), anti-IL-17 biologics (including but not limited to Secukinumab/Cosentyx), Ixekizumab/Taltz), Brodalumab/Siliq), anti-IL-23 biologics (including but not limited to Guselkumab/Tremfya), anti-IL-12/23 biologics (including but not limited to Ustekinumab/Stelara), anti-IL-5 biologics (including but not limited to Mepolizumab/Nucala, Reslizumab/Cinqair, Benralizumab/Fasenra), anti-IL-4/IL-23 biologics (including but not limited to Dupilumab/Dupixent), biologics targeting IgE (including but not limited to Omalizumab/Xolair), agents targeting lymphocyte movement/trafficking (including but not limited to Vedolizumab/Entyvio), small molecule inhibitors of any biological activity that is associated with any autoimmune disease, disorder, or condition, including but not limited to JAK inhibitors (such as but not limited to Tofacitinib/Xeljanz, Upadacitinib/Rinvoq, and Baricitinib/Olumiant), or any combination of any of these agents or any pharmaceutically acceptable salt or derivative thereof. See U.S. Pat. No. 10,092,584, the entire disclosure of which is incorporated herein by reference.

As such, the presently disclosed subject matter also relates in some embodiments to methods for treating an inflammatory and/or an autoimmune disease, disorder, or condition. In some embodiments, the inflammatory and/or an autoimmune disease, disorder, or condition is selected from the group consisting of fatty liver disease, endometriosis, types 1 and 2 diabetes, inflammatory bowel disease, asthma, obesity, Alzheimer's and Parkinson's diseases, Ankylosing Spondylitis (AS), Antiphospholipid Antibody Syndrome (APS), Gout, Inflammatory Arthritis Center, Myositis, Rheumatoid Arthritis, Scleroderma, Sjogren's Syndrome, Systemic Lupus Erythematosus (SLE, Lupus), vasculitis, Addison's disease, Celiac disease-sprue (gluten-sensitive enteropathy), dermatomyositis, Grave's disease, Hashimoto's thyroiditis, Multiple sclerosis, Myasthenia gravis, Pernicious anemia, Reactive arthritis, Rheumatoid arthritis, Psoriasis/psoriatic arthritis, multiple sclerosis, Sjögren's syndrome, Systemic lupus erythematosus (SLE), type 1 diabetes, Inflammatory bowel disease (including Crohn's disease and ulcerative colitis), autoimmune vasculitis, Guillain-Barre syndrome, and Chronic inflammatory demyelinating polyneuropathy. In some embodiments, the presently disclosed methods comprise administering to a subject in need thereof a composition of the presently disclosed subject matter in combination with at least one additional therapeutically active agent. In some embodiments, the at least one additional therapeutically active agent is an anti-inflammatory and/or an immunosuppressant agent.

The presently disclosed subject matter also relates in some embodiments to methods for treating inflammatory and/or autoimmune diseases, disorders, and/or conditions associated with sensitivity to a histone deacetylase inhibitor (HDACi). In some embodiments, the method comprises, consists essentially of, or consists of administering to a subject in need thereof an effective amount of a composition as disclosed herein. In some embodiments, the inflammatory and/or an autoimmune disease, disorder, or condition is selected from the group consisting of fatty liver disease, endometriosis, types 1 and 2 diabetes, inflammatory bowel disease, asthma, obesity, Alzheimer's and Parkinson's diseases, Ankylosing Spondylitis (AS), Antiphospholipid Antibody Syndrome (APS), Gout, Inflammatory Arthritis Center, Myositis, Rheumatoid Arthritis, Scleroderma, Sjogren's Syndrome, Systemic Lupus Erythematosus (SLE, Lupus), vasculitis, Addison's disease, Celiac disease-sprue (gluten-sensitive enteropathy), dermatomyositis, Grave's disease, Hashimoto's thyroiditis, Multiple sclerosis, Myasthenia gravis, Pernicious anemia, Reactive arthritis, Psoriasis/psoriatic arthritis, multiple sclerosis, Systemic lupus erythematosus (SLE), type 1 diabetes, Inflammatory bowel disease (including Crohn's disease and ulcerative colitis), autoimmune vasculitis, Guillain-Barre syndrome, and Chronic inflammatory demyelinating polyneuropathy.

In some embodiments, the presently disclosed methods further comprise, consist essentially of, or consist of administering to the subject at least one additional therapeutically active agent. In some embodiments, the at least one additional therapeutically active agent is an anti-inflammatory and/or an immunosuppressant agent.

### III.E. Nanoparticle Synthesis

In some embodiments, the presently disclosed subject matter provides methods for synthesizing nanoparticles, the useful approach in the methods, and the nanoparticles (NPs) made by the method and the composition thereof. In some embodiments, the presently disclosed subject matter relates to the synthesis of a nanoparticle comprising a histone deacetylase inhibitor, in some embodiments, romidepsin. In some embodiments, the method described herein involves utilizing a multi-channel derived controlled addition of a phase to a phase in a “high throughput parallel manner” to optimize NP properties on a multi-point stirrer. The particles are formed at the interface of the two solutions. In some embodiments, the method is semiautomatic and generates monodispersed NPs with well-defined morphology. The method herein involves mixing of one or more materials that form nanoparticles with a second solution or anti-solvent. The population of particles produced using the methods have a range of uniform sizes and shapes. The particles produced in accordance with some embodiments of the presently disclosed method can achieve relatively precise desired concentrations of romidepsin. In some embodiments, the produced particles have negative zeta potential. In some embodiments, the method created herein is an iterative, rapidly optimizing, low-cost fabrication technique to generate stable and scalable formulations. In some embodiments, the method describes highly effective reproducible scale-up synthesis.

As such, the presently disclosed subject matter also relates in some embodiments to methods for fabricating nanoparticles comprising one or more drug molecules. In some embodiments, the methods comprise, consist essentially of, or consist of: (a) varying in one or more iterations two or more parameters of a first or subsequent reaction mixture comprising a drug molecule and one or more polymers; (b) selecting a desired combination of parameters for a further reaction mixture based on the varying of step (a); and (c) precipitating a nanoparticle comprising the drug molecule from the further reaction mixture. In some embodiments, the reaction mixture further comprises a reaction mixture selected from the group consisting of a solvent, a non-solvent, a surfactant, and combinations thereof. In some embodiments, the solvent is an organic solvent. In some embodiments, the non-solvent is an aqueous solvent, water, or PBS buffer.

In some embodiments, the particles are prepared from polymers. The polymeric materials can be biocompatible and biodegradable. In some embodiments, the composition comprising a methoxy poly (ethylene glycol)-b-poly(D,L-Lactide), methoxy poly(ethylene glycol)-b-poly(lactic-co-glycolic acid) di-block co-polymers. In some embodiments, the population of produced particles have a monomethyl polyethylene glycol (methoxy PEG, or mPEG). In some embodiments, the core forming hydrophobic polymer chain lengths and molecular weights are variable. In some embodiments, the shell forming hydrophilic polyethylene glycol ratios are variable and controlled. It is often considered that the formulation containing polyethylene glycol protects the particles from aggregation and “opsonization”. It is often desirable to produce biocompatible particles with “stealth” properties. In some embodiments, a stealth property involves evading the immune system, metabolic routes of clearance, and the like. In some embodiments, the nanoparticle composition produced using this (these) method(s) is stable and comprises a nonionic surfactant, such as poloxamer 188. The compositions may have properties which facilitate improved pharmacologic disposition and pharmacokinetic features, toxicity and efficacy. In some embodiments, the presently disclosed subject matter provides a detailed method for the synthesis and optimization of a romidepsin polymer nanoparticle formulation for therapeutic use.

To address challenges associated with the established physio-chemical properties and toxicities associated with romidepsin, the presently disclosed subject matter provides in some embodiments an optimized nanotechnology approach to synthesize versions of the drug with improved pharmacologic behavior. These approaches can allow for a highly loading and release of the drug in an efficient prescribed manner. The unique properties of nanoparticles, such as their small size, large surface-to-volume ratios, the ability to create rational combinatorial nanotherapeutics, and the ability to bioconjugate honing motifs on their surface, provides many advantages over traditional small molecule drug design and discovery.

Based on the properties of nanoparticles and the methodology described herein, a nanoparticle comprising romidepsin (embodiments of which are referred to herein as “NanoRomidepsin” and “NanoRomi”) produces a nanotherapeutic with substantially improved drug properties, rendering it a more advantageous drug for the treatment of disease. The presently disclosed subject matter relates in some embodiments to novel polymer nanoparticle formulated with romidepsin. The formulation of the nanoparticle has been engineered to facilitate the expected desired size, encapsulation efficiency, and pharmacokinetic parameters. One or more of the listed parameters, including some or all of the listed parameters, can play a role in influencing the nanoparticle's stability, size, polydispersity index (PDI), zeta potential, and encapsulation of drug. Exemplary parameters can include one or more of the following: PDI in a range of 0 to about 0.3, size in the range of about 30-150 nanometers, a morphology selected from the group consisting of spherical, rod, and cylindrical, optionally spherical; a zeta potential in the range of about −30 to about +30, about 50-60% encapsulation efficiency; a concentration of about 500 to about 600 g/mL; use of a PEG in a range of about 2K to about 10K; and a polymer size of about 4K to about 25K. The factors, including but not limited to the chemical nature of core-forming block polymer, molecular mass of hydrophilic block polymer, concentration of the polymer, controlled addition of solvent, ratio of solvent to anti-solvent, pH of anti-solvent, choice and percent concentration of surfactant, and the core shell nature of particles have a role on the physio-chemical properties of the nanoparticle. The presently disclosed nanoparticles have been shown to improve the pharmacokinetic features of the drug.

In some embodiments, the presently disclosed methods comprise optimization of one or more parameters selected from a group consisting of mode of phase addition, a drug/polymer ratio, a drug/surfactant ratio, solvent/anti-solvent ratio, rate of addition, and combinations thereof. In some embodiments, the drug is HDACi, optionally romidepsin. In some embodiments, the HDACi/polymer ratio ranges from about 1:10 to about 1:100 W/W, optionally 1:10 to about 1:50 W/W; the HDACi/surfactant ratio ranges from about 1:0.05 to about 1:0.2 W/W; the solvent/anti-solvent ratio ranges from about 1:10 to about 1:1, optionally wherein the anti-solvent is selected from the group consisting of water, PBS, or another ionic buffer solution; and/or the rate of addition ranges from about 10 to about 500 mL/hour, optionally about 10 to about 50 mL/hour.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Materials and Methods for the Examples

Cell lines. The T cell lymphoma cell lines HH and H9 (both Cutaneous T Cell Lymphomas; CTCL), SUP-T1 (T Cell Lymphoblastic Lymphoma) and FEPD and SUP-M2 (ALK-negative anaplastic large cell lymphoma; ALCL), were obtained from ATCC. NKL (Natural killer cell lymphoblastic leukemia/lymphoma) cell lines (Robertson et al., 1996) were also employed. The cutaneous melanoma cell line FM3-29 was obtained from DSMZ. The Large Granular Lymphocyte (LGL) leukemia cell line TL-1 was generated in the Loughran lab. All cells were grown at 37° C. and 5% CO₂ in a humidified incubator. Cell lines were authenticated by short tandem repeat DNA profiling (Genetica DNA laboratories) and tested for mycoplasma contamination routinely using the MycoAlert PLUS detection kit (Lonza #LT07-710). Experiments were performed within 6 weeks of thawing. HH, H9, SUP-T1, FEPD and FM3-29 cells were cultured in RPMI-1640 (Corning, Glendale, AZ) with 10% FBS (Thermofisher Scientific, Waltham, MA). SUP-M2 cells were cultured in RPMI-1640 with 20% FBS. TL-1 cells were cultured in RPMI-1640 with 10% FBS and supplemented with 200 U/ml IL-2 (Miltenyi Biotec cat #130-097-743). NKL cells were cultured in RPMI-1640 with 10% FBS and supplemented with 100 U/ml IL-2.

Compounds. Romidepsin (depsipeptide, FK228, FR901228, NSC630176) was purchased from eNovation Chemicals (Cat #05342; New Jersey). Lipids, 1,2-distearoyl-sn-glycero-3-phosphocholine (DSPC), dioleoyl-sn-glycero-3-phosphoethanolamine (DOPE), and 1,2-dioleoylsn-glycero-3-phosphoethanolamine-N-[methoxy(polyethyleneglycol)-2000](ammonium salt; PEG2000 PE) were ordered from Avanti Polar lipids. Cholesterol and Polaxemer 188 were ordered from Sigma, all organic solvents Amicon filters, Sartorius Minisart syringe filters and general supplies were ordered from Fisher Scientific, and Sepharose CL-4B in 20% ethanol were purchased from GE Health Care, Inc. All polymers were ordered from Polyscitech, a division of Akina Inc.

Preparation and Characterization of NanoRomidepsin Liposomes. Lipid thin films were prepared by evaporating chloroform solution of lipids at 25 mg/mL and romidepsin at 1 mg/mL concentration. Neutral liposomes were prepared by mixing in the following molar ratio: the order of the lipids DSPC:DOPE:PEG2000PE and romidepsin in the 5.67:2.83:1.0:0.5. Neutral+5% cholesterol formulations were then mixed in the following molar ratio; 5.3:2.67:1.0:0.5:0.5, respectively, to a total volume of 1 mL of lipids. Romidepsin was dissolved in chloroform at concentration of 1 mg/mL, and 0.5 M ratio solution was added to the lipid sample. The lipids mixtures were thoroughly mixed in a glass test tube and then the chloroform was evaporated under nitrogen at 35-40° C. to complete dryness (forms a thin film around the test tube) for 2-3 hours. Trace amounts of chloroform were removed under reduced pressure via rotary evaporator for 30 minutes. Next, the liposomes were rehydrated with 1×PBS in a heat shaker for 2 hours (60° C.; 600 rpm), vortexing every 15 minutes to convert into micelles and synthesized into liposomes, which were then placed in a sonic bath for 20 minutes to break up and homogenize the liposome samples.

The liposomes were then sized using an Avanti Mini extruder (Avanti Polar Lipids) fitted with a 0.1 μm polycarbonate membrane and by passing liposomal solution back and forth between the syringes 11 times. The final sample was collected and then purified by a size exclusion column over sepharose beads using 1×PBS as an eluent to remove free drug.

### Preparation and Characterization of NanoRomidepsin Polymer Particles.

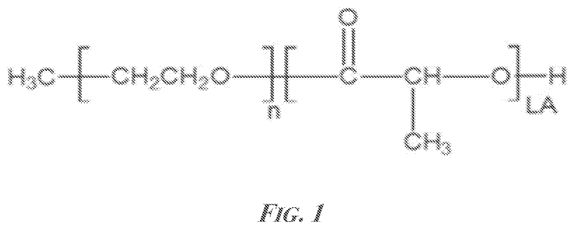

NanoRomidepsin Polymer Particles were prepared by bulk nanoprecipitation. Acetonitrile solutions of mPEG-PDLLA Polymer (FIG. 1), romidepsin, and polaxamer 188 were prepared at 10 mg/mL, 2 mg/mL, and 1 mg/ML concentrations. 0.5 mL of each, Polymer and drug solutions (Drug/Polymer ratio; 1/5), and 0.1 mL surfactant (10% of w/w to the drug) were mixed and stirred continuously at 500 rpm. Milli Q nanopure water (Organic/aqueous ratio; 1:9) was added at 10 ml/hour rate. The solvent dispersion was recorded over the period and the stirring continued for three more hours to remove the organic solvent completely. Finally, nanoparticles were extracted by centrifugation at 2000 rpm for 20 minutes at 24° C. The particles were collected by reconstituting into nanopure water or 1×PBS. NanoRomidepsin particles were stored at 4° C. These formulations' particle size and morphology were evaluated using DLS and Cryo-EM. Drug concentrations were determined on analytical Mass spectrometry, LC-MS as described below.

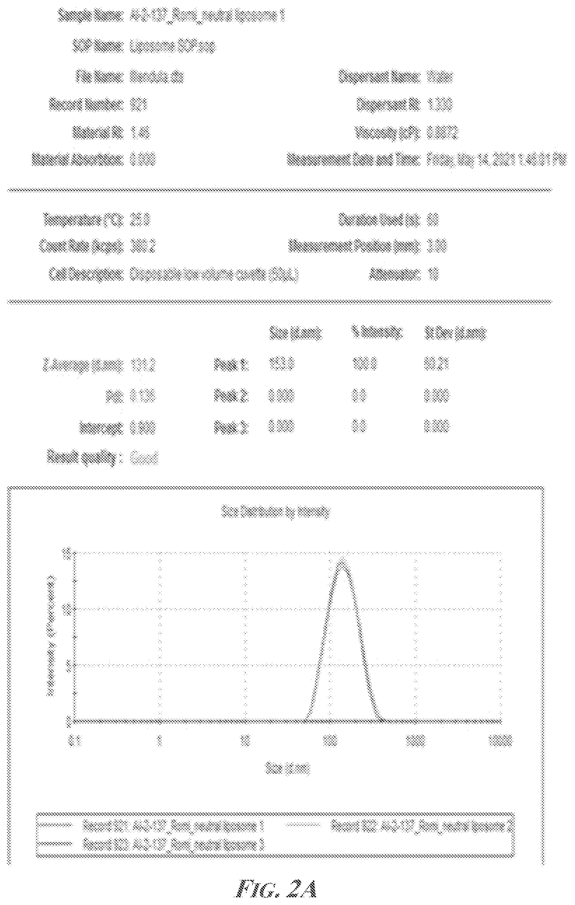

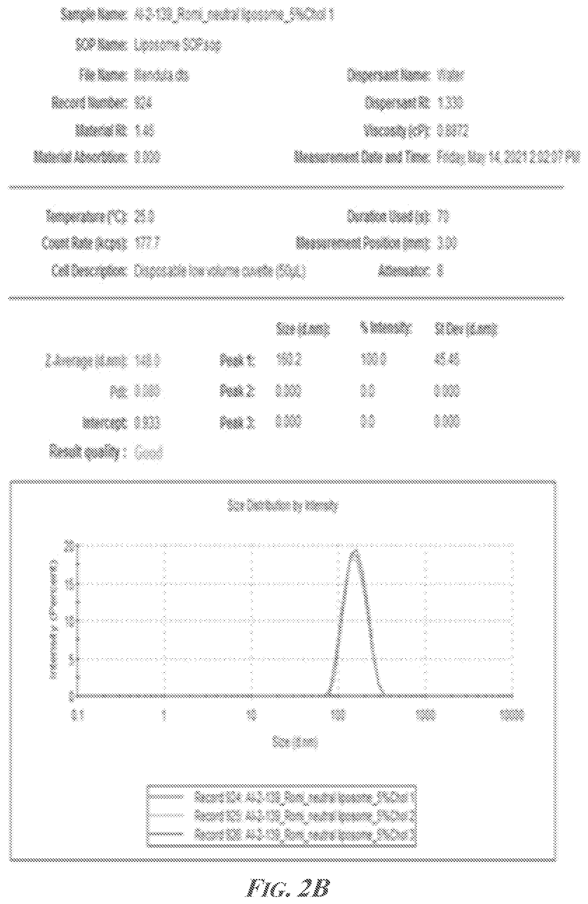

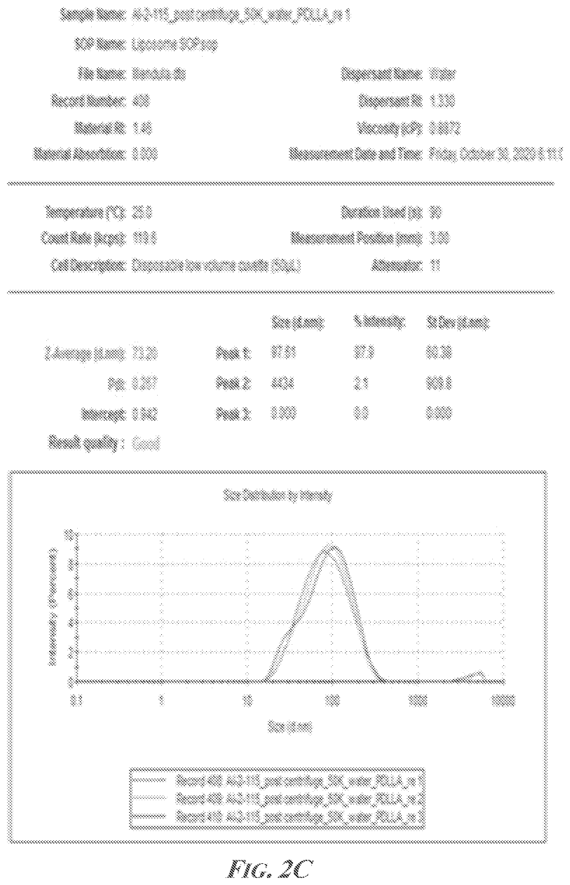

Dynamic Light Scattering (DLS). DLS measurements were performed on polymer nanoparticles. Size distribution of the particles was analyzed by dynamic light scattering using a Zetasizer (Malvern Instruments model ZEN 3690, Malvern, Worcestershire, WR141XZ, United Kingdom). Percentage of particles of a specific size versus particle diameter was measured three times at 25° C. (see FIGS. 2A-2C).

Electron Microscopy. The size and morphology of polymer particles were measured using a FEI Tecnai F20 (FEI, Hillsboro, OR) transmission electron microscope operating at 120 kV cryo-Electron microscopy (cryo-EM). The cryo-EM samples were prepared by a standard verification method. An aliquot of ˜3 μl sample solution was applied onto a glow-discharged perforated carbon-coated grid, (2/1-3C C-Flat; Protochips, Raleigh, NC, USA) and the excess solution was blotted with filter paper. The samples were then quickly plunged into a reservoir of liquid ethane at −180° C. The vitrified samples were stored in liquid nitrogen and transferred to a Gatan 626 cryogenic sample holder (Gatan, Pleasontville, CA) and then maintained in the microscope at −180° C. All images were recorded with a Gatan 4K×4K pixel CCD camera under cryo-condition at a magnification of 9600× or 29,000× with a pixel size of 1.12 nm or 0.37 nm, respectively, at the specimen level, and at a nominal defocus ranging from −1 to −3 μm. The particles recorded at 29,000×, and images were recorded at 50 nm scale bar. Exemplary images are shown in FIG. 3.

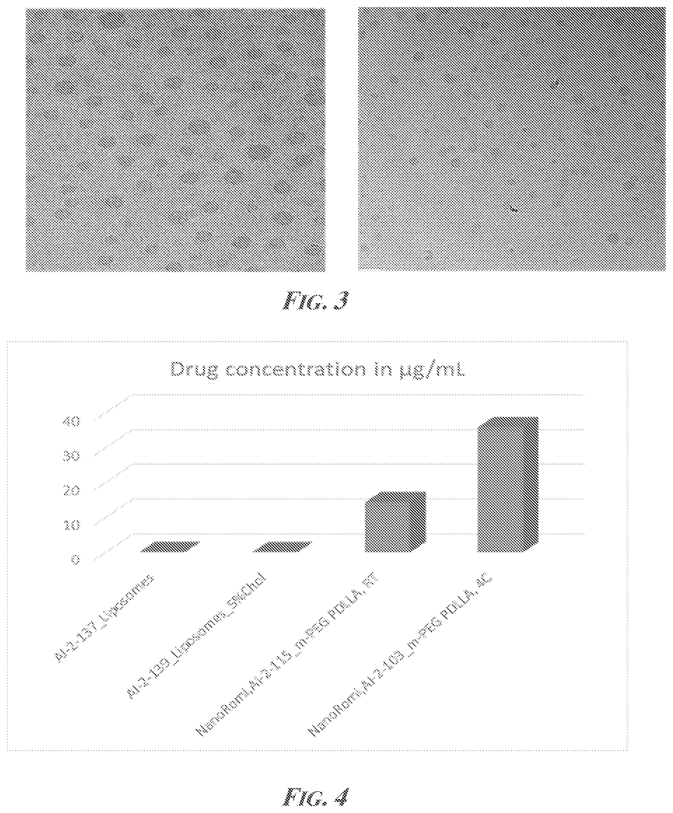

LC-MS Quantification of Romidepsin in Nanoparticles. LC-MS was performed on TQ-S spectrometer (Waters Corporation, Milford, MA). Samples were analyzed by passing through Acquity C18 column BEH hybrid technology with the particle size 1.7 μm and column ID 2.1 mm×50 mm length. Column temperature was set to 50° C., injection volume of 1 μL, and eluted with a gradient of mobile phase A: Water+0.1% Formic Acid and mobile phase B: MeOH+0.1% Formic Acid at the flow rate of 0.5 mL/min for 2 minutes. Exemplary results are shown in FIG. 4.

Cell Viability Assay. For cell viability, cell lines were plated at the appropriate cell densities (SUP-T1, HH, NKL and TL-1 at 100,000 cells/ml/well and H9, FEPD SUP-M2 and FM3-29 at 50,000 cells/ml/well) in a 48-well plate. NanoRomidepsin mPEG PDLLA PBS, NanoRomidepsin mPEG PDLLA H₂O, NanoRomidepsin mPEG PLGA H₂O, or unencapsulated romidepsin were added to the cells at concentrations ranging from 0.03 nM to 30 nm. Empty ghost Nano mPEG PDLLA and Nano mPEG PLGA were added to the various cell lines as a standard control at varying dilutions in order to achieve the same concentrations of the polymer encapsulated romidepsin. Cells were harvested after 24, 60, and 96 hours of incubation at 37° C., 5% CO₂ and assayed for cell viability, using CELLTITER-GLO® (Promega) luminescence reagent, by quantifying the amount of ATP present. After 15 minutes of adding CELLTITER-GLO® reagent at a 1:1 ratio, luminescence was read on a GLOMAX® Discover Microplate Reader (Promega). Luminescence was normalized to untreated control, which was defined as 100% viability.

Flow Cytometry. Cells were harvested after 30 hours of treatment, washed twice with PBS, fixed with 4% paraformaldehyde solution and permeabilized with 70% methanol before incubating with the relevant antibodies: HDAC1 Polyclonal Antibody (Proteintech), Rabbit anti-Acetyl Histone H3 K9/K14, and Rabbit Anti-Acetyl Histone H4 (Millipore). After 30 minutes incubation at room temperature cells are washed twice with PBS and FITC conjugated secondary Goat anti-rabbit antibodies (Thermofisher) were added. Cells were incubated on ice for 30 minutes and then washed in PBS. After the second wash, cells were resuspended in PBS with 4% FBS and ready for signal acquisition on a flow cytometer. Apoptosis in cell lines was assessed by the presence of cleaved Poly (ADP-ribose) polymerase-1 (PARP) using a flourochrome conjugated anti-cleaved PARP antibody (BD Biosciences). After staining the cells were acquired using an Attune Flow Cytometer and the data analyzed using FloJo analysis software. The Mean Fluorescence intensity (MFI) was indicative of the level of expression of each marker.

Western Blots. Cells were incubated with the indicated concentrations for each drug and nano polymer under normal growth conditions for 24 hours. Proteins from total cell lysates were resolved on 12% to 20% SDS-PAGE and transferred to nitrocellulose membranes. Membranes were blocked in phospho-buffered saline and 0.05% Triton X-100 containing 5% skim milk powder, and were then probed overnight with specific primary antibodies. Antibodies were detected with the corresponding horseradish peroxidase-linked secondary antibodies. Blots were developed using Bio-Rad CLARITY™ and CLARITY MAX™ ECL chemiluminescent substrate detection reagents. Signal detection and imaging was captured and analyzed using a CHEMIDOC™ System (Bio-Rad) with signal quantification. The monoclonal and polyclonal antibodies used were as follows: acetylated histone H3, acetylated histone H4, vinculin, and (3-actin (all from Cell Signaling Technology).

In vivo studies. For single dose toxicity study, five to seven-week old female BALB/c mice were chosen for the MTD study. Animals were randomly assigned to dose groups based on body weight. Prior to assignment to groups, the weight variation of the animals did not exceed 20% of the mean weight. Mice were divided into two cohorts for Nano-romidepsin (NanoRomi) and free romidepsin. Each cohort was divided into sub-cohorts. Each sub cohort (n=5) received a single treatment of following doses: 1, 2, 3, 5, and 8 mg/kg body weight by intreaperitoneal (IP) and intravenous (IV) route of administration. NanoRomi was diluted under sterile conditions in phosphate buffered saline (PBS). Romidepsin was prepared in DMSO and then diluted with PBS under sterile condition. For repeat dose MTD, the mice were treated with 2 and 3 mg/kg of NanoRomidepsin PDLLA and free romidepsin for 1, 4, 8 and 11 days intraperitoneally. The mice were monitored for 7 days after the last treatment. For single dose and repeated dose toxicity study, we used two endpoints: weight loss and clinical score. Clinical signs were scored by observing activity, appearance (hair coat and eyes/nose), posture, and body condition with a maximum of 3 points going to each (0, normal; 1 slight deviation from normal; 2, moderate deviation from normal, 3, severe deviation from normal). Euthanasia criteria included weight loss either >20% weight loss or reached a clinical score >6.

For the pharmacokinetic study, mice were divided into two treatment cohorts, one for NanoRomidepsin PDLLA and the other for free romidepsin. Each treatment cohort was further divided into two sub-cohorts depending upon the route of administration (IP or IV). Each sub-cohort (n=21) received a single treatment of ½ MTD as defined from our single dose toxicity study (2.5 mg/kg body weight) of NanoRomi or free romidepsin. Mice were sacrificed (n=3 per time point) at 1, 3, 6, 18, 24, 48, and 72 hours after the treatment. Blood (˜1 mL) was collected via terminal cardiac puncture under CO₂ anesthesia and collected in EDTA-coated K3EDTA tubes followed by centrifugation (2000×g for 15 min) to isolate plasma. Plasma was placed in cryopreservation vials and preserved by snap freezing using liquid nitrogen. Blood from three untreated mice was collected at the beginning of the treatment (T=0) and used as a control. The level of romidepsin was quantified by a validated method based on reversed-phase liquid chromatography coupled to tandem mass-spectrometric detection with a standard curve derived with stock romidepsin.

Data analysis: The pharmacokinetic profile of free romidepsin/NanoRomidepsin PDLLA in plasma were analyzed by non-compartmental analysis using PKSolver software to determine the pharmacokinetics parameters such as maximum concentration (Cmax), time of Cmax (Tmax), area under curve (AUC) and half-life. For efficacy study: 5- to 7-week-old female NOD.Cg-Prkdcscid Il2rgtm1Wjl/SzJ mice (The Jackson Laboratory). were injected with 3 million a fluorescent protein, Chili (dTomato-absorption max and emission max at 554 nm and 581 nm respectively)—and a bioluminescence generating protein, firefly luciferase (Luc) expressing H9 cells subcutaneously in the flank. In vivo BLI analysis was conducted on a cryogenically cooled Lago X (Spectral Instruments Imaging system). Mice were imaged twice a week starting 4 days after inoculation of cells. Once xenograft tumors reached over bioluminescence intensity (photon/s) over >107, mice were randomized to 4 treatment groups of 10 animals each: (i) control group treated with normal PBS alone; (ii) Ghost Nanoparticle (iii) romidepsin (2 mg/kg) (iv) NanoRomidepsin (2 mg/kg) were administered by intraperitoneal injection on days 1, 4, 8, and 11. Baseline imaging data were recorded for all mice on day 1 (start of drug administration) and on each day of drug administration before the treatment. The numbers of animals, study design, and treatment of animals were reviewed, and approved by the Institutional Animal Care and Use Committee (IACUC) of the University of Virginia, Charlottesville, Virginia, United States of America.

### Example 1

### Encapsulation of Romidepsin in Polymer Nanoparticles

We initially chose a liposomal Nano formulation to encapsulate romidepsin. Liposomes are widely accepted nanocarriers due to their significant contributions toward improving cellular and tissue uptake and compound bio-distribution in vivo. We prepared a neutral liposomal formulation of romidepsin by using standard lipid combinations, which in some embodiments included DSPC, DOPE, and/or PEG. The method yielded nanoparticles with acceptable sizes, 131 nM and 148 nM and a polydispersity index (PDI) of 0.14 and 0.08 in neutral liposomes and cholesterol liposomes, respectively. However, the concentration of the drug in the particles was very poor (less than 1%) or negligible even after addition of cholesterol to the lipids while preparing the liposomes. Given these non-optimal results, we continued to re-engineer the nanoparticles.

Polymer nanoparticles have drawn increased attention over the past several years in cancer research as well as targeting drug delivery. FDA approved biodegradable, amphiphilic block co-polymers, poly lactic-co-glycolic acid (PLGA) to poly dl-lactic acid (PDLLA) polymers play a critical role in creation of biocompatible NPs. We thus continued our efforts, chose m-PEG PDLLA with molecular weight (Mw˜5,000:10,000 Da), and prepared Romidepsin polymer nanoparticles by bottom-up “bulk nanoprecipitation or solvent displacement” method. The hydrophobicity of this di-block co-polymer may favor the entrapment of hydrophobic drugs. We developed a bulk nanoprecipitation method using a specific Drug/Polymer/Surfactant ratio. Systemic experimental operating conditions were included in the design of the experiment by varying drug/polymer (D/P) ratio, organic to water (O/W) ratio, drug/surfactant (D/S) ratio, temperature, and choice of solvents to obtain optimal size and PDI and improve the drug concentration in the particles. Higher drug loading was achieved at D/P; 1:5, O/W; 1:9; D/polaxamer188 1:0.1 (w/w) 4° C. The method yielded nanoparticles with a reasonable poly-dispersity index (PDI) between 0.24 to 0.26 and a z-average of 60-70 nM. Other types of polymers including higher molecular weight of PDLLA, keeping PEG at 5K, (5K:20K) PEG to Polymer, various ratios of m-PEG and PLGA including (2K:10K, 5K:20K, 5K:45K, 5K:75K keeping LA:GA ratio at 50:50), and m-PEG-PCL with various surfactants and D/P ratio at RT and 4° C. are investigated to improve drug concentration for in vivo experiments. The above formulation/nanoparticles were stable at 4° C. for more than a year. A model Nano polymer scaffold, NanoRomidepsin, with optimal encapsulation of romidepsin was achieved that provides sustained release kinetics.

### Example 2

### Nanoromidepsin Polymers Reduce Cell Viability and Induce Apoptosis in Cancer Cells

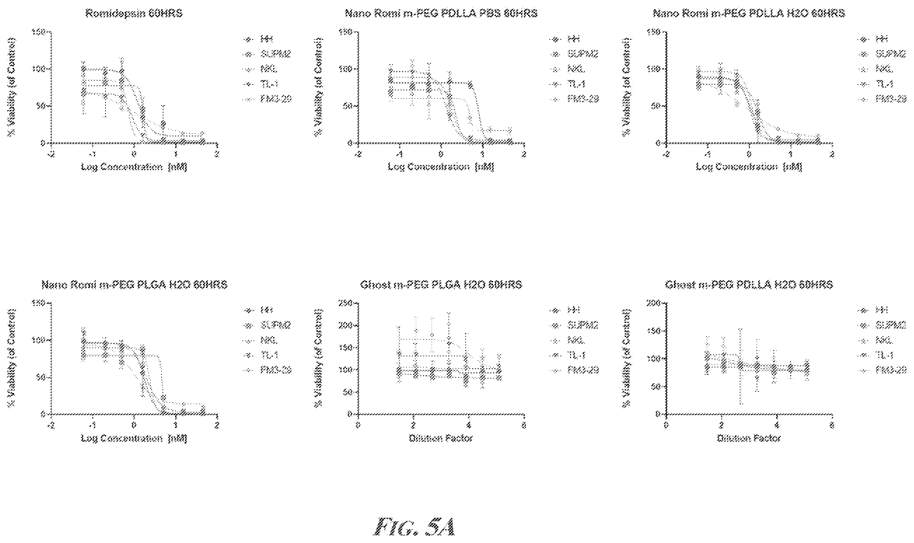

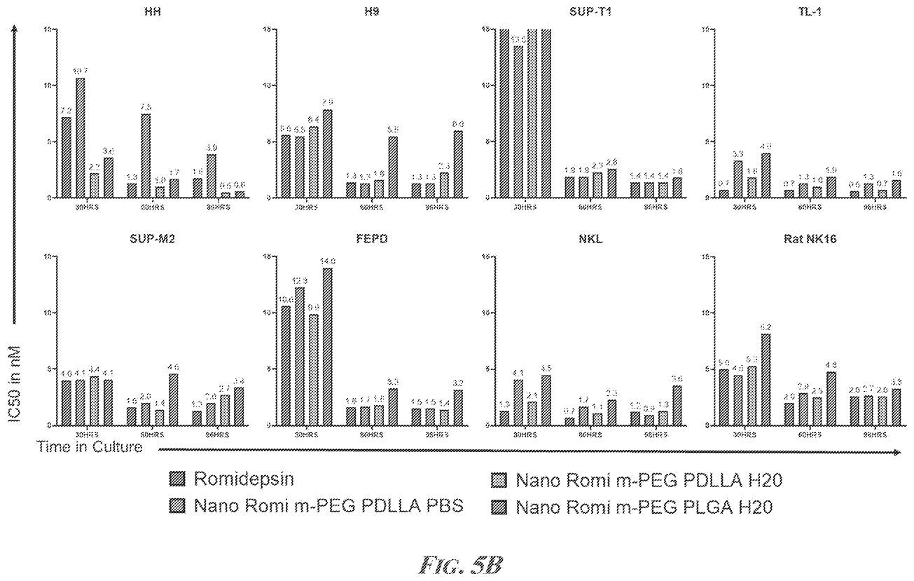

A comparative concentration-response assessment was performed to identify the pattern of response on cell viability following treatment with romidepsin and the three nano polymers containing romidepsin in the cancer cell lines HH and H9 (CTCL), SUP-T1 (T Cell Lymphoblastic Lymphoma), FEPD (ALCL), SUP-M2 (ALK-ALCL), NKL (Natural killer cell lymphoblastic leukemia/lymphoma) TL-1 (LGL leukemia) and FM3-29 (cutaneous melanoma). The IC₅₀ (inhibiting 50% of proliferative capabilities) was determined following analysis of an increasing range of doses from 0.3-30 nM romidepsin and NanoRomidepsin polymers at 30, 60 and 96 hrs. (FIGS. 5A and 5B). Empty ghost polymers were diluted accordingly. All 3 formulations of NanoRomidepsin inhibited cell growth of all the cancer cell lines in a concentration and time-dependent manner. (FIG. 5B). The inhibitory capacity of 50% viability (IC₅₀) varied for the three nano formulations for the various cell lines tested. At 60 hours, the cancer cell lines consistently were most sensitive to NanoRomidepsin mPEG PDLLA H₂O (IC₅₀ in 0.7-1.9 nM range; Avg. 1.2), which was very similar to romidepsin (IC₅₀ in 0.6-1.9 nM range; Avg. 1.225; Table 2). Both NanoRomidepsin mPEG PDLLA PBS (IC₅₀ in nM range 1.3-7.5; Avg. 2.812; Table 2) and Nano mPEG PLGA (IC₅₀ in nM range 1.1-5.5; Avg. 2.875; Table 2) were less potent as compared to romidepsin and NanoRomidepsin mPEG PDLLA H₂O. There was no inhibition of the growth of any of cancer cell lines tested when the corresponding empty ghost Nano particles were surveyed (FIG. 5A). A comparison of the cytotoxic activity of the three NanoRomidepsin drugs to romidepsin in a spontaneous leukemic cell line NKL-16 from F344 rats with structural and functional features of rat NK cells, showed similar to human cancer cell lines, NanoRomidepsin mPEG PDLLA H₂O was the most potent of the three formulations with IC₅₀s very similar to romidepsin at all three time points (24, 48, and 72 hrs) tested (FIG. 5B).

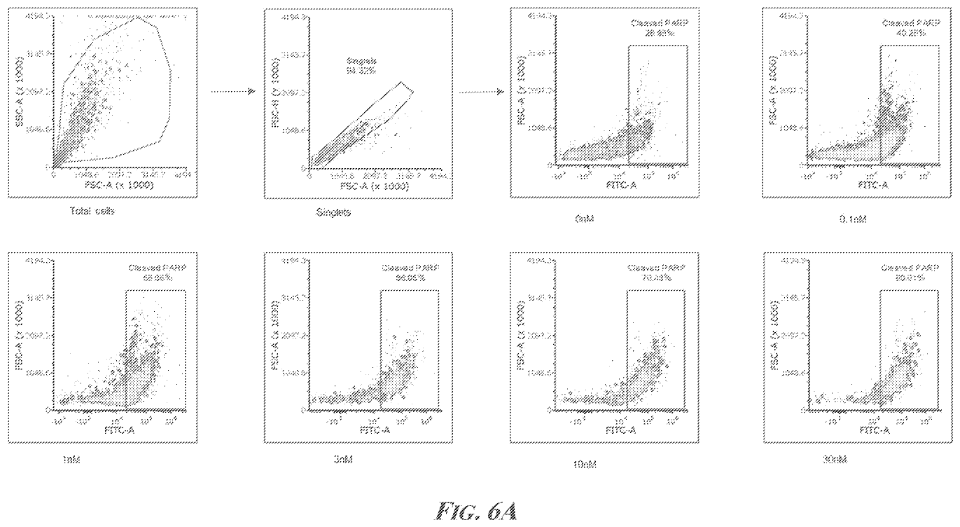

Flow cytometry demonstrated that treatment with all the three nano polymers induced apoptosis similar to romidepsin as shown by an increase in the expression of cleaved PARP (FIGS. 6A and 6B). However, NanoRomidepsin mPEG-PDLLA PBS and NanoRomidepsin m-PEG PDLLA H₂O showed increased PARP cleavage compared to the romidepsin and NanoRomidepsin m-PEG-PLGA.

### Example 3

### NanoRomidepsin PDLLA was the Most Potent Romidepsin Nano-Polymer Across all Cell Lines

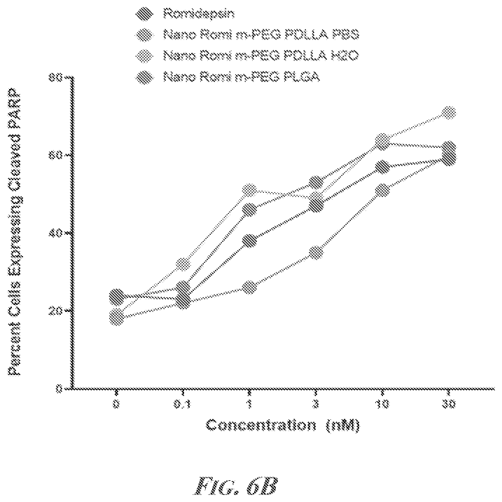

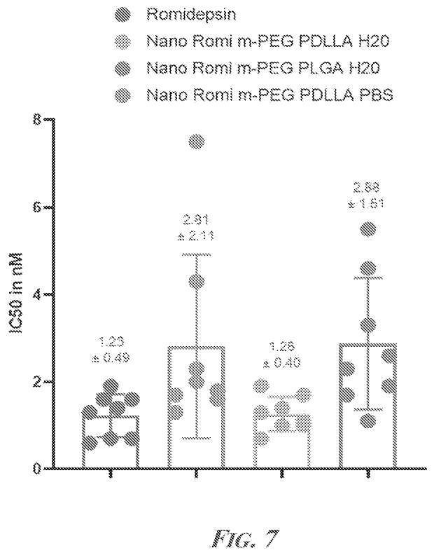

From the flow cytometry analysis of apoptosis and cell viability assays, it was evident that all the three nano polymers exhibited significant cell death and induction of apoptosis in cancer cell lines in a disease and histology independent manner. Across all the cancer cell lines, at 60 hrs, Nano-Romi mPEG PDLLA H₂O showed the lowest IC₅₀ value among all the three NanoRomidepsin polymers and the IC₅₀ value (1.26+/−0.40 nM) was comparable to romidepsin (1.23+/−0.49 nM) and significantly less (p<0.002) less than the IC₅₀ value of Nano-Romi mPEG PLGA H₂O (2.88+/−1.51 nM; FIG. 7). Based on this, we selected mPEG PDLLA as the candidate nano polymer for subsequent studies.

### Example 4

### Nanoromidepsin PDLLA Induces Acetylation of Histone Proteins in Cancer Cells

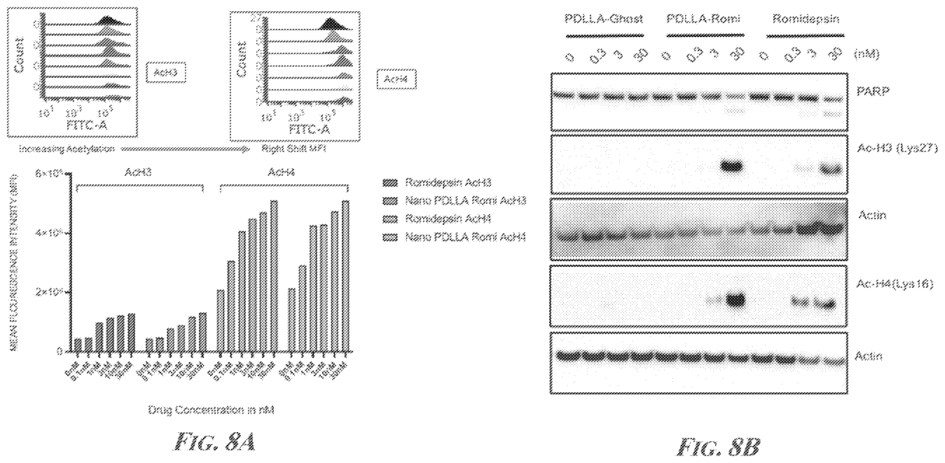

Lysine acetylation is a reversible post-translational modification of proteins and plays a key role in regulating gene expression (Choudhary et al., 2009). Romidepsin is relatively selective for class I histone deacetylase enzymes, HDAC1 and HDAC2, that increases acetylation of lysine residues on histone 3 (H3) and histone 4 (H4; Furumai et al., 2002; Luchenko et al., 2014). The levels of acetylation on H3 and H4 were measured following treatment with increasing doses of romidepsin and NanoRomidepsin PDLLA. There was a concentration dependent increase in the acetylation of both H3 and H4 observed using flow cytometry when cancer cells were treated with either romidepsin or NanoRomidepsin PDLLA at 30 hours (FIG. 8A). The levels of acetylation on H3 lysine 27 and H4 lysine 16 were measured following treatment with increasing doses of romidepsin and NanoRomidepsin PDLLA. Western blot analysis shows the increase in acetylation of H3 and H4 following treatment with 0.3 to 30 nM of romidepsin or NanoRomidepsin PDLLA at 30 hours (FIG. 8B). The increased acetylation of both H3 and H4 proteins was 2 to 3-fold more in the cells treated with 30 nm NanoRomidepsin PDLLA compared to those treated with 30 nM romidepsin.

### Example 5

Susceptibilities of Normal Cells from Healthy Donors and Cancer Cells to NanoRomidepsin PDLLA and Free Romidepsin

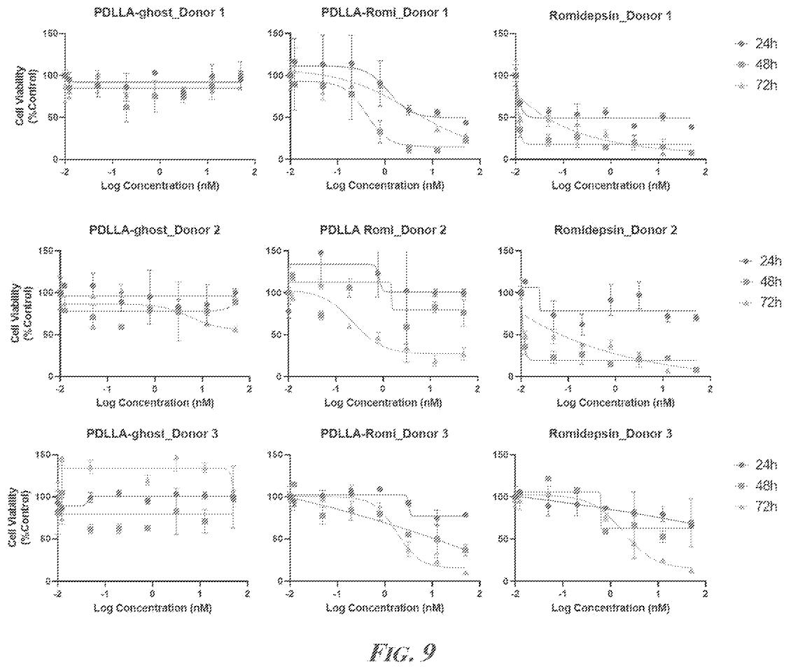

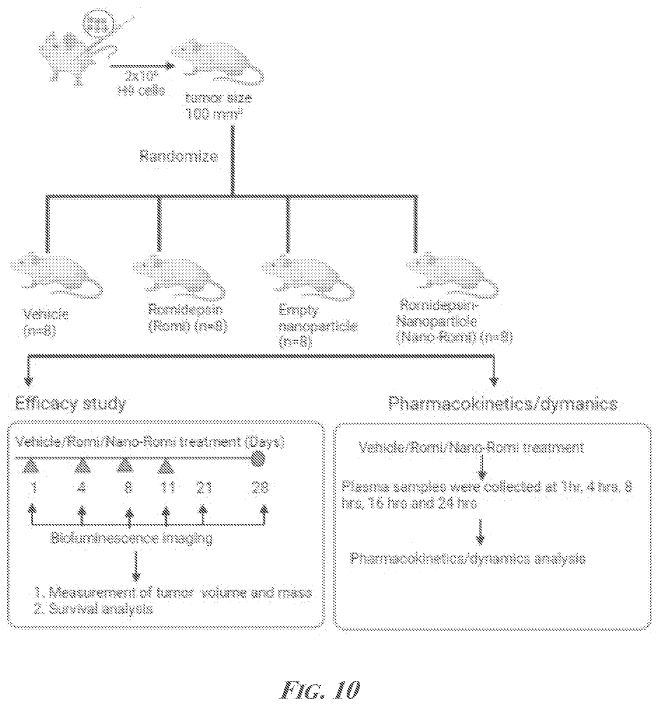

Peripheral blood mononuclear cells (PBMCs) from three healthy donors were cultured and treated with increasing concentration of romidepsin, NanoRomidepsin PDLLA, or Ghost PDLLA polymer (i.e., PDLLA polymer without romidepsin) for 24, 48 and 72 hours. The cells from all 3 donors were susceptible to both romidepsin and NanoRomidepsin PDLLA, but not the Ghost PDLLA polymer, in a time and dose dependent manner (FIG. 9). However, at 48 and 72 hrs, PBMCs from donors 1 and 2 were a range of 4 to 150-fold less sensitive to NanoRomidepsin PDLLA, than to romidepsin (Table 3). At 72 hours, PBMCs from donor 3 were equally sensitive to both romidepsin (IC₅₀ 2.48) and NanoRomidepsin PDLLA (IC₅₀ 2.48; Table 3). At 60 hrs the NanoRomidepsin PDLLA was 2.4× more potent across a range of cancer cells (IC₅₀ in 0.7-1.9 nM range; Avg. 1.2; Table 3) than across the PBMCs from healthy donors (IC₅₀ in 0.4-6.3 nM range; Avg. 3.0) while at 72 hrs the PBMCs from healthy donors were 1.5× more sensitive to romidpsin (IC₅₀ in 0.04-2.5 nM range; Avg. 0.8) than the cancer cells IC₅₀ in 0.6-1.9 nM range; Avg. 1.225; Table 2).

### Example 6

### In Vivo Studies

The effects of NanoRomidepsin PDLLA in an in vivo mouse model of cancer, initially lymphoma, were investigated. Single and multiple-dose toxicity studies were performed to identify the maximum tolerated dose (MTD) and dosing schedules for future efficacy studies. Pharmacokinetic study was performed to investigate the half-life, bio-distribution and release kinetic profile of the NanoRomidepsin polymer.

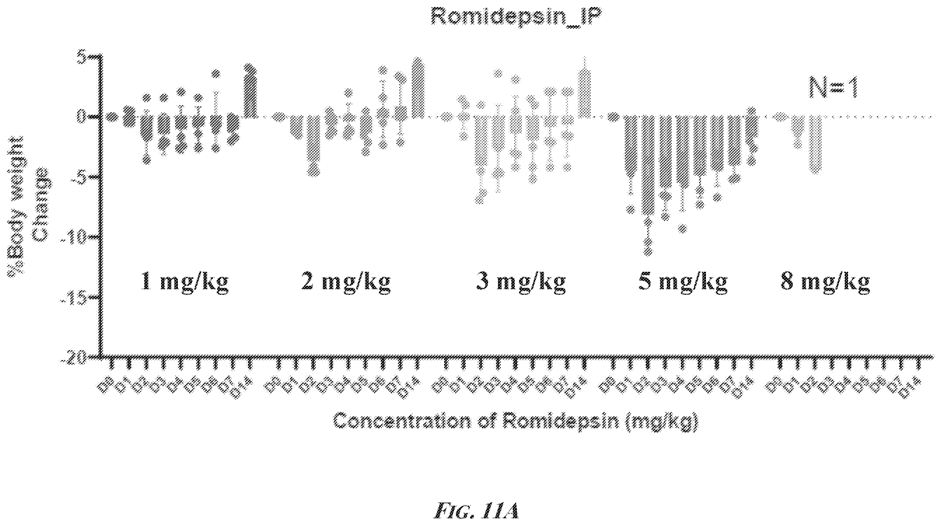

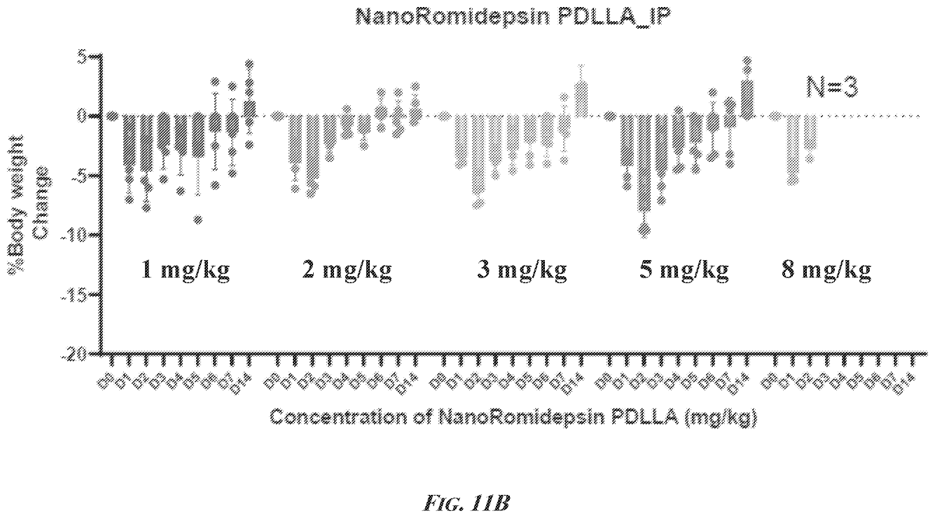

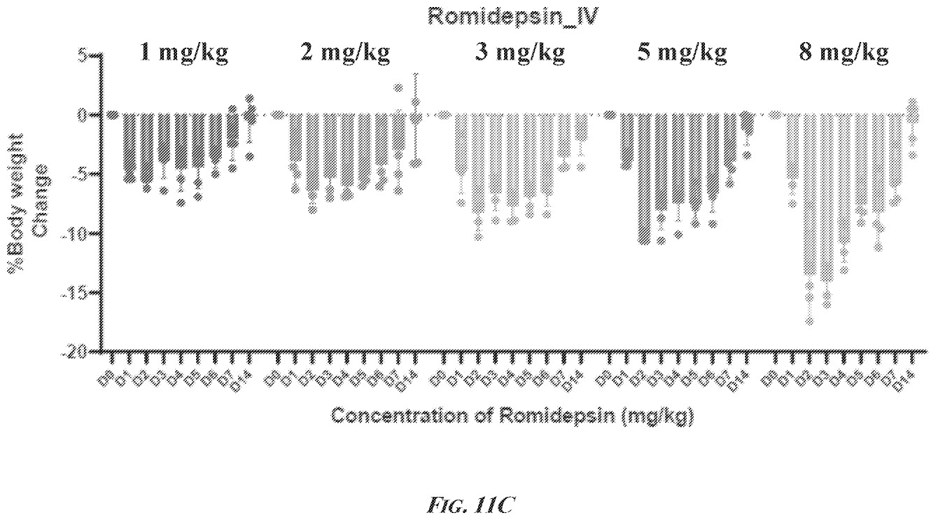

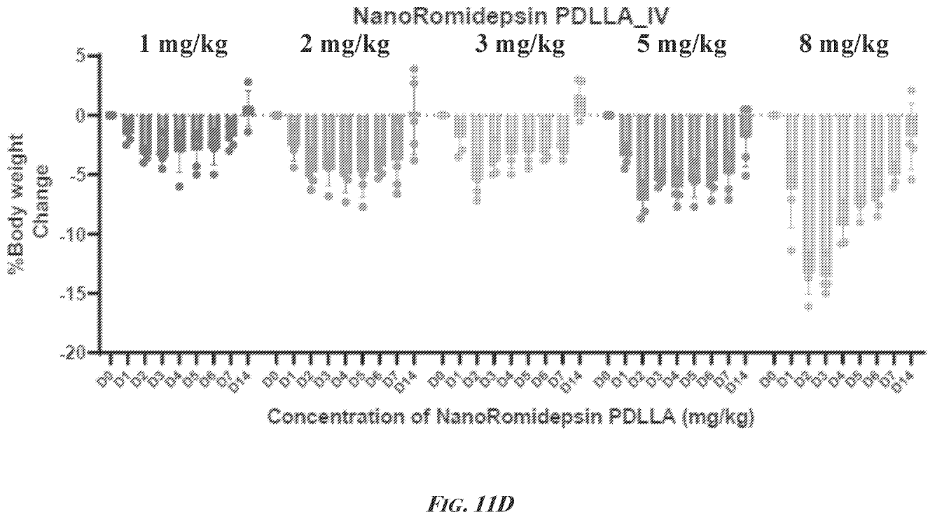

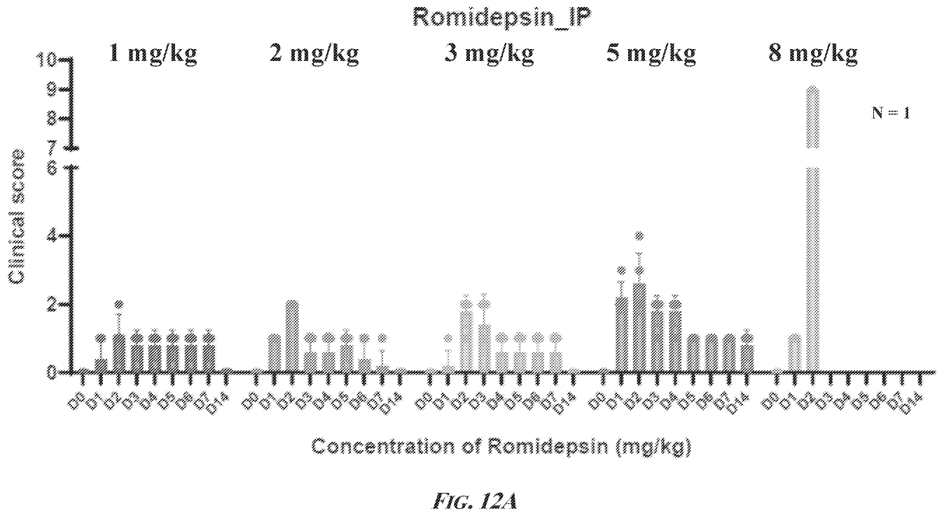

Single dose toxicity assay: A clear correlation was observed between dose and weight loss and/or clinical score in both free romidepsin and NanoRomidepsin PDLLA treated groups. Both romidepsin and NanoRomidepsin PDLLA caused acute weight loss that returned to baseline within 14 days (FIGS. 11A-11D). Mice reached humane endpoints at 8 mg/kg (upper panel) for intraperitoneal (IP) route of administration for both drugs. For the intraperitoneal (IP) route of administration, the MTD was determined 5 mg/kg for romidepsin and NanoRomidepsin PDLLA. For the intravenous (IV) route of administration, the mice were only treated until 8 mg/kg with romidepsin and NanoRomidepsin PDLLA (lower panel). However, we did not reach the MTD for IV treatment with 8 mg/kg dose of romidpesin. The single dose toxicity study suggested that NanoRomidepsin PDLLA showed almost equivalent toxicity compared to the free romidepsin. In addition, both romidepsin and NanoRomi showed equivalent clinical score after first 7 days of treatment (FIGS. 12A-12D).

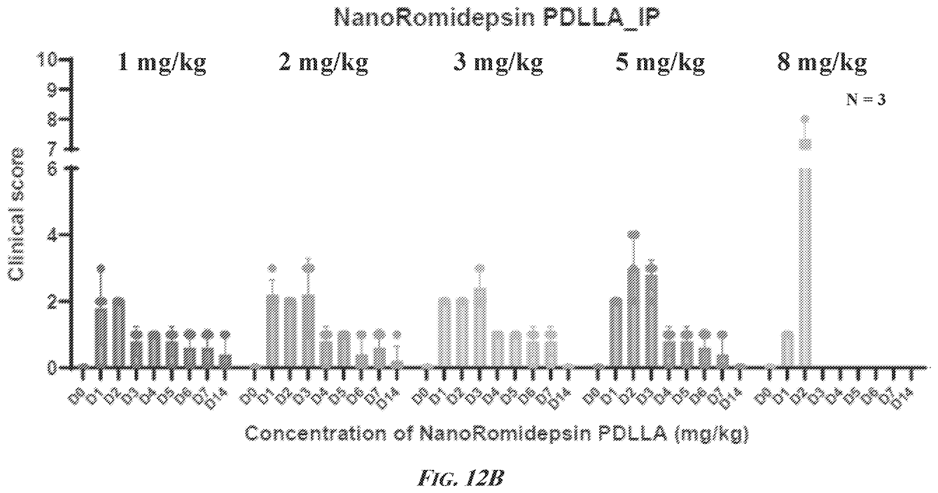

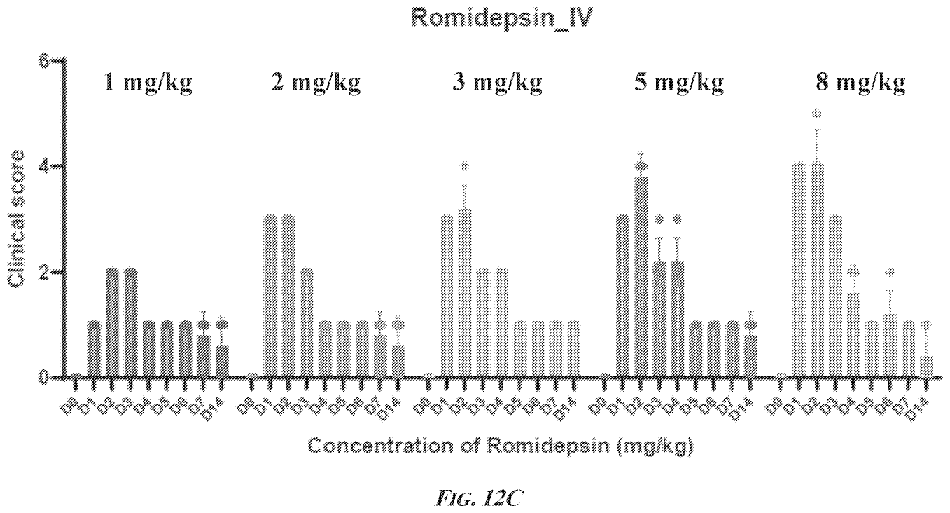

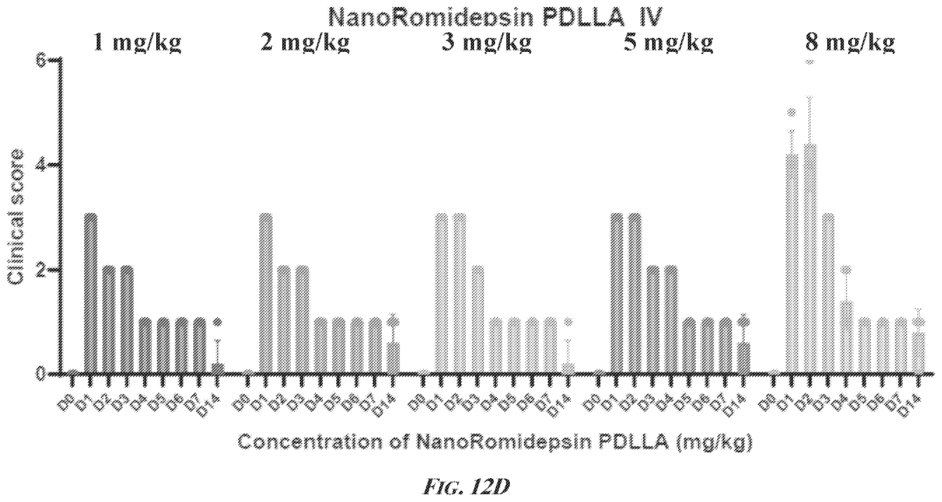

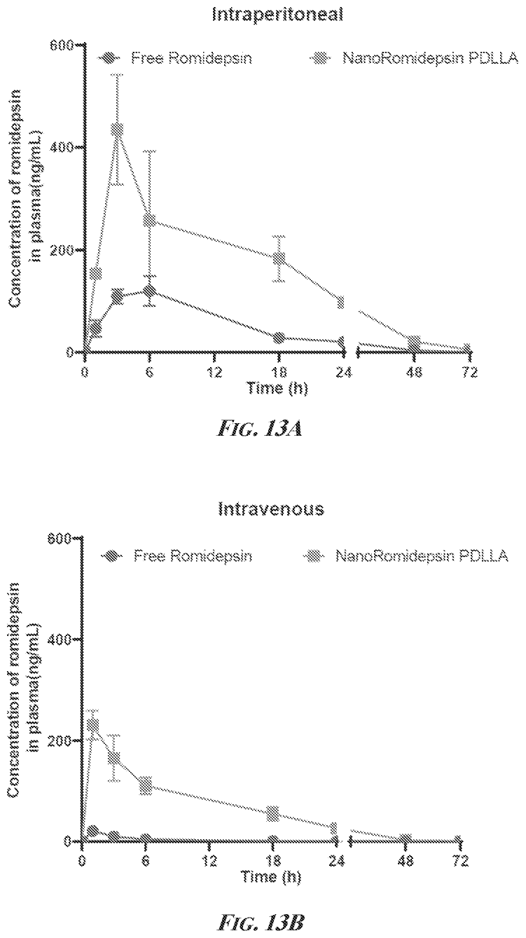

Pharmacokinetics analysis: Pharmacokinetic (PK) analyses were performed to investigate the preferred route of administration and dose for the repeated dose toxicity study and efficacy study. In the PK study, the concentration of romidepsin in plasma after free romidepsin/NanoRomidepsin PDLLA treatment by the IP route (FIG. 13A) was higher compared to the same dose given by the IV route (FIG. 13B; free romidepsin IP>free romidepsin IV; NanoRomi IP>NanoRomi IV). The PK analyses indicated that the clearance of romidepsin from the plasma by the IV route was faster compared to the IP route of administration. NanoRomidepsin PDLLA delivered by IP route showed higher plasma romidepsin concentration (Cmax) and Tmax compared to the free formulation (Table 4). The Cmax and Area under the curve (AUC) for NanoRomidepsin PDLLA were 3.5-fold higher compared to the free romidepsin for the IP route. For IV route, the Cmax and AUC for NanoRomidepsin PDLLA are 10-fold and 25-fold higher compared to the free romidepsin (Table 3). However, the AUC for NanoRomi is three-fold higher in the IP route compared to the IV route. Based on this data, IP route was selected for the preferred route of dose administration for future studies. The peak concentration of romidepsin after administration of NanoRomidepsin PDLLA and free romidepsin by IP were 804 nM and 218 nM, respectively. For the IV route, the peak concentration of NanoRomidepsin PDLLA and free romidepsin were 425 nM and 38 nM, respectively. According to our in vitro data in T cell lymphoma cell lines, the EC₅₀ of NanoRomidepsin PDLLA is around 2-8 nM. PK analysis suggests that we achieved the 80-400 fold of EC₅₀ concentration in the plasma with one-half MTD dose administration.

Repeated dose toxicity assay: Next, repeat dose toxicity assay was performed to identify the appropriate dose of NanoRomidepsin PDLLA for the efficacy study. Mice were administered 2 and 3 mg/kg free romidepsin/NanoRomi for 1, 4, 8, and 11 days and monitored until the mice completely recovered. From the pharmacokinetics analysis, intraperitoneal route was chosen as the route of administration. During the experimental period, no deaths were observed in both 2 and 3 mg/kg free romidepsin/NanoRomidepsin PDLLA treated group (except 1 mouse in free romidepsin treated group). In 2 mg/kg free romidepsin/NanoRomi treated groups, <10% loss in body weight (upper panel) and slight changes in haircoat and activity (lower panel) were observed in both romidepsin and NanoRomi treated groups. However, all the mice were well-tolerated with the 2 mg/kg dose of free-romidepsin/NanoRomidepsin PDLLA over 11 days of treatment and recovered their normal weight after 2-5 days of post-treatment (FIGS. 14A and 14B). However, 3 mg/kg romidepsin/NanoRomidepsin PDLLA treated group showed severe body weight loss and clinical score>4 and were not recommended for repeated dose toxicity study. In addition, the allometry calculation showed that the human equivalent dose of 2 mg/kg NanoRomidepsinPDLLA/free romidpesin is 0.16 mg/kg and the total amount of free romidepsin/NanoRomi administered in repeated dose toxicity assay is 0.64 mg/kg which is comparable to the romidepsin treatment (1.1 mg/kg) over days 1, 8, and 15 of a 28-day cycle in human adults lymphoma patients.

Efficacy study analysis: Mice inoculated with dtomato luciferase-expressing H9 cells were randomized to one of four groups of treatment (n=10 in each cohort): (i) control group treated with normal PBS alone; (ii) Ghost Nanoparticle (iii) romidepsin (2 mg/kg) (iv) NanoRomidepsin (2 mg/kg) were administered by intraperitoneal injection on days 1, 4, 8, and 11 days. Mice were imaged on day 7, day 11, day 14, and day 17 after the engraftment of H9 cell line. The mice will be monitored over the next few weeks to monitor the tumor growth in the control vs treatment group. After day 17 of engraftment, a cytostatic effect was observed in the romidepsin and Nanoromidepsin PDLLA group compared to the control and ghost nanoparticle group (FIG. 15). As of Day 17 post-inoculation, no significant difference in the treatment response was observed between romidepsin and Nanoromidepsin treated groups, although previous literature had suggested that the optimal treatment response for romidepsin usually occurs approximately 21 days after treatment. We expect that the dose-response between romidepsin and Nanoromidepsin treated groups would be more conclusive over the ensuing weeks of follow-up. The efficacy study is expanded to include other models of T-cell lymphoma, including the Angioimmunoblastic T-cell lymphoma mice model, and patient-derived peripheral T cell lymphoma xenograft (PDX) mouse model.

### Example 7

Development of Combination Nanotherapeutics with HDAC Inhibitors and Complementary Drug Partners

Romidepsin and other HDAC inhibitors have been shown to synergize with a host of other targeted drugs including hypomethylating agents, pralatrexate, and biologics (see e.g., Kalac et al., 2011; Amengual et al., 2013; Jain et al., 2015; Marchi et al., 2015; Lue et al., 2016; O'Connor et al., 2019; Falchi et al., 2021; Scotto et al., 2021). Synergism between histone deacetylase inhibitors and hypomethylating agents in preclinical PTCL models has been shown (Marchi et al., 2015; Scotto et al., 2021), and Phase 1 and Pase 2 studies of oral 5-azacytidine (AZA) and romidepsin (ROMI) in patients with advanced lymphoid malignancies were conducted and supported the contention in human patients. The combination was substantially more active in patients with PTCL than in those with non-T cell lymphoma. These studies established that combined epigenetic modifiers are potently active in PTCL patients (O'Connor et al., 2019; Falchi et al., 2021).

Combinatorial nanotherapeutics predicated on a HDAC inhibitor partner are undertaken. These combinations are employed to augment the monotherapy activity, and to prime tumor cells and the tumor microenvironment for other immunologic drugs including but not limited to checkpoint inhibitors (e.g., CTLA4, PD-1, and/or PDL-1 inhibitors). Based on detailed pharmacology focused on the development of ratiometric combinations across a battery of assets known to complement HDAC inhibitors, libraries of combination nanotherapeutics are developed to be used for the treatment of cancer and autoimmune disorders.

### Materials and Methods for Example 8

Materials. m-PEG-PDLLA 5,000:10,000 Da, m-PEG-PDLLA 5000:4000 Da, m-PEG-PDLLA 5000:14000 Da m-PEG-PDLLA 5000:20000 Da, PLGA-PEG 5,000:20,000 Da were purchased from PolySciTech®. Romidepsin was from eNovation Chemicals (Cat #05342; New Jersey). Poloxamer was obtained from Sigma Aldrich. Solvents: Acetonitrile (ACN), Acetone+10% Dimethyl Sulfoxide (DMSO), Tetrahydrofuran (THF), Ethanol (EtOH), Chloroform+Methanol (Me-OH) (1:1) were purchased from Fisher Scientific, and Centrifugation filters were purchased from Amicon (10K, 50K, 100K). The Chemyx Fusion 200 Syringe Pump was used with dual channel or multichannel syringes.

### Preparation, Optimization, Scale-Up and Characterization of NanoRomidepsin Polymer Particles.

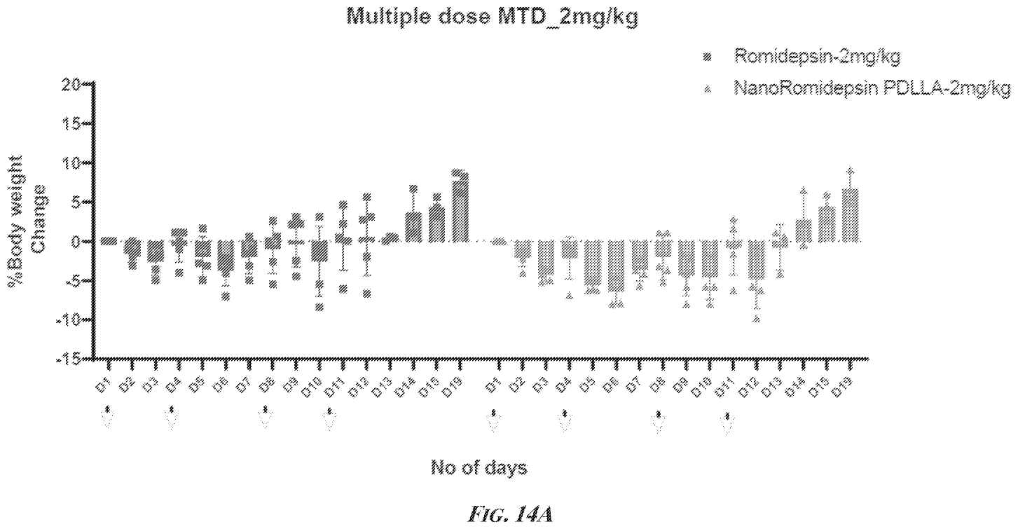

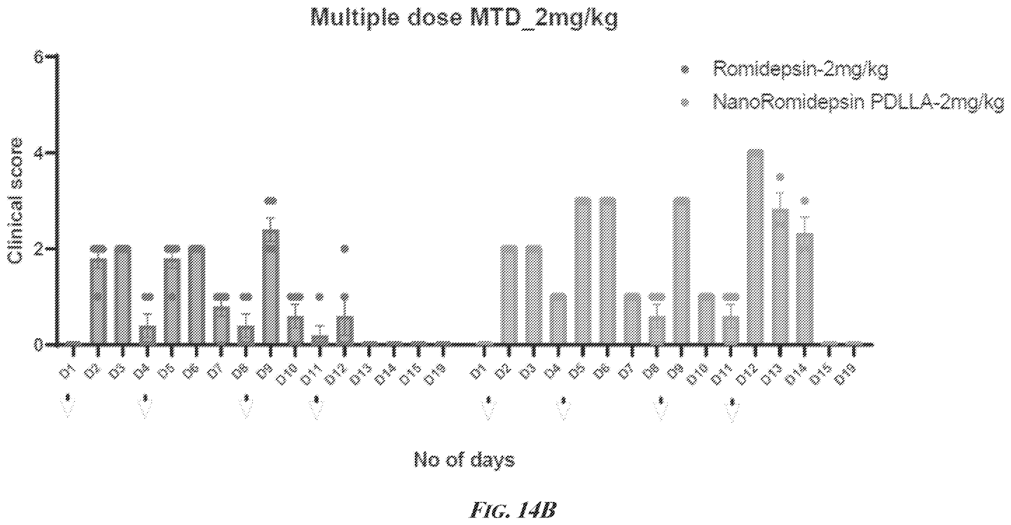

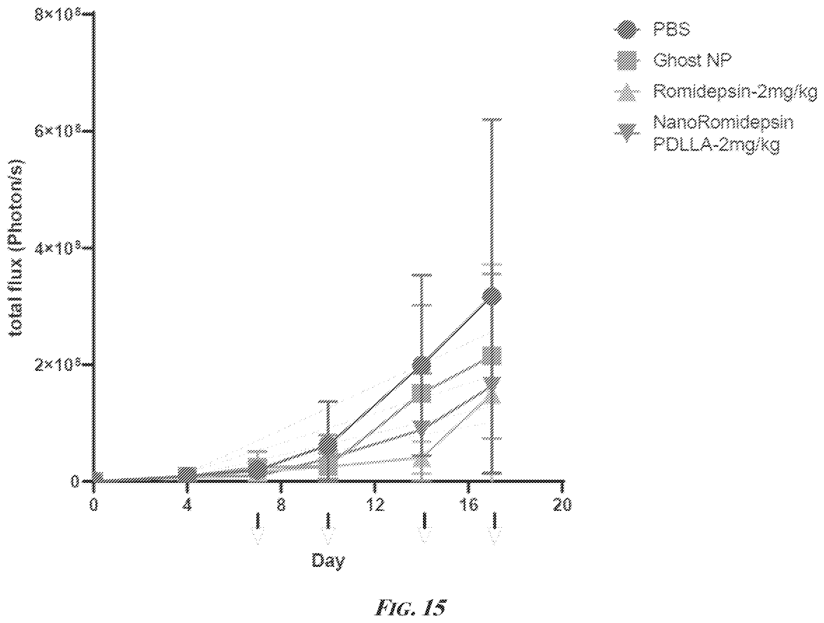

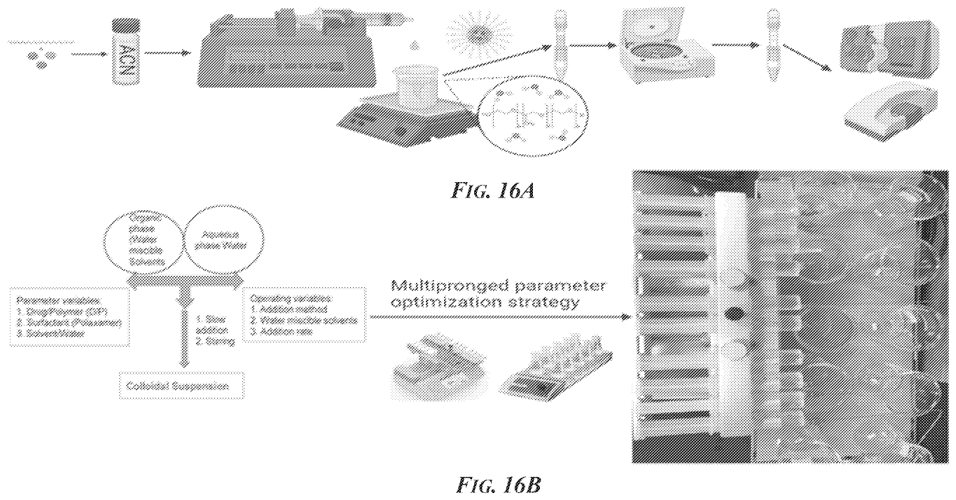

Preparation. NanoRomidepsin polymer particles were prepared by bulk nanoprecipitation. Acetonitrile solutions of mPEG-PDLLA Polymer (see FIG. 16A), romidepsin, and polaxamer 188 were prepared at 10 mg/mL, 2 mg/mL, and 1 mg/ML concentrations respectively. 0.5 mL of each, polymer and drug solutions (Drug/Polymer ratio; 1/5), and 0.1 mL surfactant (10% of w/w to the drug) were mixed and stirred continuously at 500 rpm. Milli Q nanopure water (Organic/aqueous ratio; 1:9) was added at 10 ml/hour rate. The solvent dispersion was recorded over the period and the stirring continued for three more hours to remove the organic solvent completely. Finally, nanoparticles were extracted by centrifugation at 2000 rpm for 20 minutes at 24° C. The particles were collected by reconstituting into nanopure water or 1×PBS. NanoRomidepsin particles were stored at 4° C. These formulations' particle size and morphology were evaluated using DLS and Cryo-EM. Drug concentrations were determined on analytical Mass spectrometry, LC-MS as described below.

Optimization of drug concentration of NanoRomidepsin formulation. Optimization of NanoRomidepsin formulation was achieved by an iterative experimental approach, by using an engineered semi-automated syringe pump to deliver a phase by controlled addition and nanoprecipitation method. Thus, the formulation parameters and operating parameters (FIG. 16B) were sequentially modified to manufacture the NPs with improved drug concentration. The sequentially altered parameters included but were not limited to drug/polymer ratio, drug to surfactant ratio, polymer length, hydrophilic PEG to core forming hydrophobic polymer ratio, polymer choice, solvent screening, solvent/anti-solvent ratio, mixing speed, rates, variation of phase to phase addition, processing speeds, and filters to achieve a desired particle size, PDI, and drug concentration in the NPs. During each parameter optimization, a group of 2-10 variables are included, making the design of the experiment parallel. The best out of those variables was chosen and incorporated into the next parameter optimization, further making it an iterative operation.

Solvent Screening: Role of solvent on NanoRomdepsin NPs by Nanoprecipitation method. A m-PEG PDLLA 5:10 kDa stock solution was made in ACN. To probe the solvent effects on NP formation, a group of five water-miscible solvents, ACN, Acetone+10% DMSO, THF, EtOH, Chloroform+Me-OH (1:1)) were considered for the preparation of Romidepsin NPs and drug solutions were made in these solvents respectively, and vortexed to dissolve. Drug and polymer solutions were combined to achieve a drug to polymer ratio of 1:25. A Poloxamer solution was prepared by dissolving it in ACN, at 10% with respect to the drug mass. A syringe pump was used for phase distribution at a predetermined rate of 50 mL/hr with constant surfactant, concentration, and solvent/anti-solvent ratio. The solution was stirred for 3 hours in a fume hood to allow evaporation of the organic phase. Centrifugation was performed with centrifugal filter units with 100K membrane cutoff to purify the sample. Nanoparticles were collected, reconstituted into water, stored at 4° C., and characterized by the above-mentioned methods, morphological properties using DLS/cryo-EM and drug concentration by LC/MS.

Polymer Optimization: Role of Co-polymer on NanoRomdepsin NPs by nanoprecipitation method. Polymer nanoparticles were prepared with two types of block co-polymers, m-PEG-PDLLA, & m-PEG-PLGA, different polymer diblock lengths of PDLLA polymer are included; m-PEG-PDLLA 5:10 kDa, m-PEG-PDLLA 5:4 kDa, m-PEG-PDLLA 5:14 kDa, m-PEG-PDLLA 5:20 kDa, m-PEG-PLGA (50:50) 5:20 kDa. m-PEG-PDLLA 5:10 kDa particles are made with drug in two-different ratio's; D/P 1:10 & D/P 1:25. Polymers were dissolved in ACN and vortexed to ensure complete dissolution. A drug stock solution was prepared in ACN. Poloxamer solution was prepared by dissolving it in ACN, using 10% of solution with respect to the drug mass. Drug and polymer solutions, at a ratio of 1:10 or 1:25, were included in optimization. A syringe pump was used for a phase distribution at a predetermined rate of 50 mL/hr. Fixed drug-to-surfactant ratio and solvent/anti-ratio were maintained. The solution was stirred for 3 hours in a fume hood to allow evaporation of the organic phase. Centrifugation was performed with centrifugal filter units with 100K membrane cutoff to purify the sample. Nanoparticles were collected, and reconstituted into water, stored at 4 C and characterized by the above-mentioned methods, morphological properties using DLS/cryo-EM and drug concentration by LC/MS.

Centrifugal Filter Optimization: Role of filter units on processing of NanoRomdepsin NPs. Drug and polymer solutions were made in ACN and vortexed to achieve solute dissolution. Solutions were mixed at a drug to polymer ratio of 1:25, and loaded into a syringe. In a scintillation vial, poloxamer was diluted in water to 10% with respect to the drug mass to achieve a drug-to-poloxamer ratio of 10:1. A syringe pump was used for phase distribution at a predetermined rate of 50 mL/hr, with a fixed solvent/anti-solvent ratio. The solution was stirred for 3 hours in a fume hood to allow evaporation of the organic phase. Centrifugation was performed with centrifugal filter units with Amicon ultra centrifugation filters 10K, 50K, and 100K membrane cutoff to separate the unencapsulated drug from the sample. In another set, we used Rhodamine dye in two different ratios and used 10K and 100K filters. Nanoparticles were collected, reconstituted into water, and stored at 4 C and characterized by the above-mentioned methods, morphological properties using DLS/cryo-EM, and drug concentration by LC/MS.

Optimization of solvent to anti-solvent ratio: Organic to Water ratio in the preparation of NanoRomdepsin NPs. Drug and m-PEG PDLLA (5K:10K) polymer solutions were made in ACN and vortexed to get a clear solution. Solutions were mixed at a drug to polymer ratio of 1:25 and loaded into syringes. In a scintillation vial, poloxamer solution was diluted in water to 10% with respect to the drug mass, to achieve a drug to poloxamer ratio of 10:1. Syringe pump is used for phase distribution at a predetermined rate of 50 mL/hr, and to distribute the pre-calculated solvent/anti-solvent ratio. Multiple experiments with various ratios of organic to water are considered; 1:9, 1:5, 1:3, 1:2, and 1:1. Recently a 1:1 ratio of organic to non-solvent, 1×PBS was also considered. The solutions were stirred for 3 hours in a fume hood to allow evaporation of the organic phase. Centrifugation was performed with centrifugal filter units with 100K membrane cutoff to purify the sample. Nanoparticles were collected, reconstituted into water, and stored at 4 C and characterized by the above-mentioned methods, morphological properties using DLS/cryo-EM, and drug concentration by LC/MS.

Scale-up of NanoRomidepsin formulations. NanoRomidepsin formulation was scaled-up to 1-20 mL by utilizing the iterative method of optimization of parameters. The best condition from each parameter optimization set with respect to drug concentration is included in scale-up. Romidepsin, m-PEG-PDLLA 5:10 kDa, and poloxamer 188 were dissolved in ACN and vortexed to ensure complete dissolution of drug and polymer in ACN to achieve predetermined ratio (D/P 1:25, D/surfactant 1:0.1 W/W). A syringe pump was used for phase distribution at a predetermined rate of 50 mL/hr. Fixed drug-to-surfactant ratio and solvent/anti-ratio were maintained. The solution was stirred for 3 hours in a fume hood to allow evaporation of the organic phase. Centrifugation was performed with centrifugal filter units with 100K membrane cutoff to purify the sample. Nanoparticles were collected, reconstituted into water, stored at 4 C, and characterized by the above-mentioned methods, morphological properties using DLS/cryo-EM, and drug concentration by LC/MS.

Characterization of NanoRomidepsin formulations. In these formulations' particle size, monodispersity, zeta potential and morphology were evaluated using DLS and Cryo-EM. Drug concentrations were determined on analytical Mass spectrometry, LC-MS as described elsewhere herein.

### Example 8

### Optimization of Drug Concentration of NanoRomidepsin Formulations

A NanoRomidepsin scaffold was developed using a bottom-up, nanoprecipitation method and continued to re-engineer the nanoparticles. The hydrophobicity of m-PEG PDLLA (5K:10K) polymer favors the entrapment of hydrophobic drugs. The traditional nanoprecipitation method is a simple, scalable, reproducible, single-step process applied for encapsulation of hydrophobic drugs (see FIG. 16A). NanoRomidepsin was shown to be equally potent as compared to equivalent concentrations of free romidepsin in in vitro assays on multiple cell lines. Scaling up the product was pursued to support in vivo experiments. In one embodiment, the highest concentration of Romidepsin in the Nano formulation was 31 g/mL, which amounted to a very low encapsulation efficiency (EE) of 1.5%. Achieving in vivo plasma concentrations for a therapeutic effect in in vivo experiments employed an iterative experimental approach to improve the EE. Particularly, a combinatorial approach for the nanoprecipitation method was developed and sequentially modified the parameters/operating conditions on the basis of the “principles of parallel synthesis” (FIG. 16B) to improve drug concentration. The NP formulation was thus prepared by using this engineered method and facilitated the proposed in vivo experiments in a short time.

The following tandem approach was demonstrated as a representative approach to improve drug concentration. In some embodiments, the nanoprecipitation method is single-step and suitable for NP property optimization in a combinatorial fashion. This Example includes the optimization of parameters considered with respect to the inclusion of hydrophobic drug, romidepsin. The expected outcome pertains to drug loading along with optimal size and PDI. The mechanism of polymer NP formation by this method involves multiple operating parameters and intrinsic properties of polymer, drug, and solvent. The controlled addition of polymer containing organic phase into aqueous phase at specific stirring rate and rate controlled/dependent mixing of phases cause diffusion of the organic solvent into the aqueous solution. As the organic solvent evaporates, the solubility of polymer decreases, and supersaturation is reached, leading to nucleation, growth, and precipitation to form NPs. To conform to the most energetically favorable structure, the amphiphilic, diblock co-polymer (in some embodiments, mPEG-PDLLA, although other amphiphilic, pegylated polymers including other PDLLAs, PLGAs, PLA, and PCL can be employed) is forced into a core-shell spherical structure making up the particle core and entrapping the drug held together by hydrophobic and other potential interactions between the drug and hydrophobic block. The water-soluble PEG arms extend into the aqueous phase forming the shell. Solvent properties dictate NP formation through directing effects. Efficient solvent-water exchange promotes NP formation. Diffusion rate of solvent in water is another factor that influences physio-chemical properties and formation of NPs, which is dependent on the solvent miscibility, ionic strength, density, viscosity, and stirring rate. Acetonitrile and acetone have high diffusion coefficients, which are known to promote NP formation. Romidepsin and polymer solubility were tested in a suitable organic solvent for nanoprecipitation method. Solubility of romidepsin and water miscibility of organic solvents with high volatility and less in vivo toxicity were the criteria in solvent screening. Parameters included in the method as well as the properties of the polymer determine the size, shape, and physio-chemical properties of the polymers NPs. The biodegradable PEG block co-polymers self-assemble into PEGylated polymeric micelles, nanospheres, or bilayer particles, and polymersomes in various sizes ranging from about 20 to about 1000 nM. The biodegradability, hydrophobicity, and other physiochemical properties of di-block polymers favors steady release of drug by surface erosion and diffusion of the core, thus the amphiphilic di-block polymers have an impact in controlled-release drug delivery applications. The commercial availability of polymers with different molecular weights and various compositions have permitted us to prepare NPs using a parallel approach which allows for a choice of nanoparticles for romidepsin drug delivery. In order to create a foundation for scale up and to understand the role of each variable on EE, multiple synthesis parameters are explored. We tested our proposed method of “parallel approach of parameters optimization”. We considered five parameters and 2-3 variables in each parameter were included in the study. We measured the effect of these parameters on a “key” NP property, which is drug loading. The list of parameters and variables included, 1. Mode of phase addition (organic into water/water into organic), 2. Drug/polymer; three variables: 1/10; 1/25; and 1/50 W/W, 3. Organic solvent/water; three variables: 1:9, 1:3, and 1:2, 4. drug/surfactant ratios; three variables: 1/0.1, 1/0.2, and 1/0.05 and 5. rate of addition three variables 20, 30, and 50 mL/hour. We identified mode of phase addition, drug/polymer ratio, and organic solvent to water ratio as key parameters that have substantial effect on the drug loading. The rate and surfactant ratio also have an effect on the drug loading, but not as significant. Using our parallel multipronged, iterative approach, five parameters were able to be optimized in a single experiment. The results from this study quickly provided the scope of the project “Go/No Go” decision. We selected the best variable from each parameter and incorporated it into one experiment, allowing to establish the cumulative effect of these parameters; addition of organic into water, D/P 1:25, Organic/Water 1:2 and 50 mL/hour rate on the drug loading. We found that the parallel approach made a significant improvement in the production of NanoRomidepsin, and made the significant impact on the drug loading optimization studies. Consequently, this strategy improved the drug concentration from the first approach with EE of 1.4% to 76.26% in the following experiment, revealing representative optimized parameters; all results are listed in (Table 5A). This aided in elucidating the role and impact of each variable independently and collectively on the NP EE. We identified these parameters as key parameters that can potentially ‘maximize the drug loading’ in nanoparticles: phase of addition, drug/polymer, drug/surfactant, rate of addition, and solvent/anti-solvent.

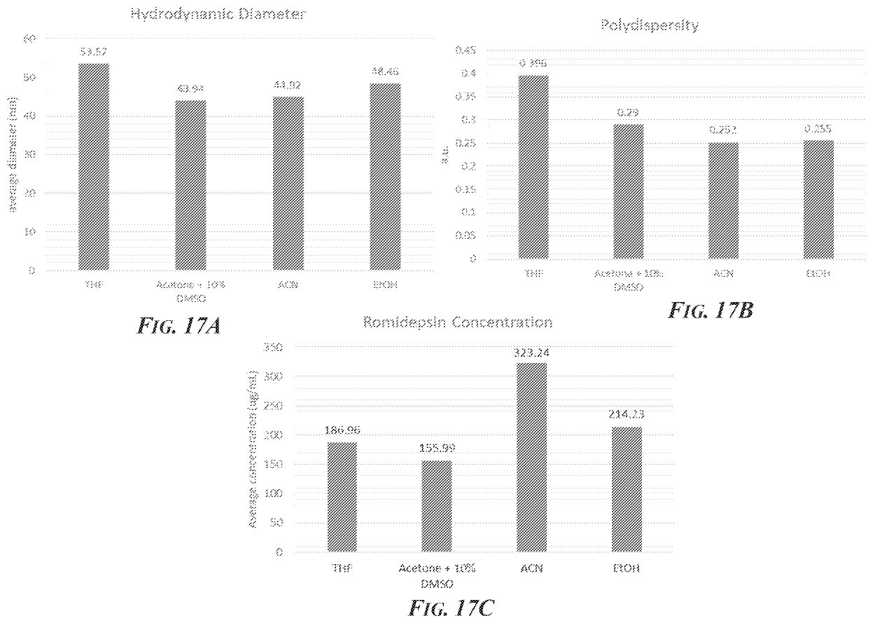

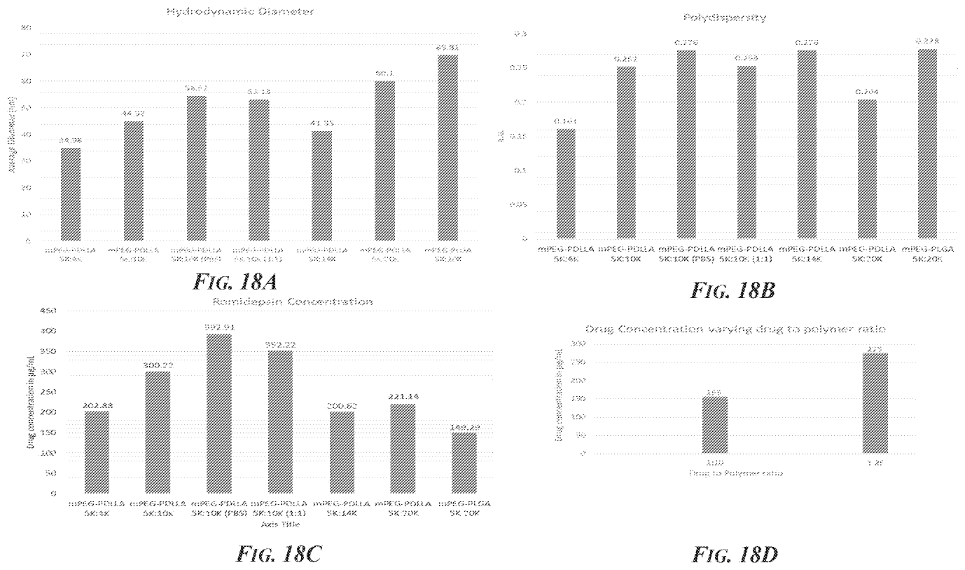

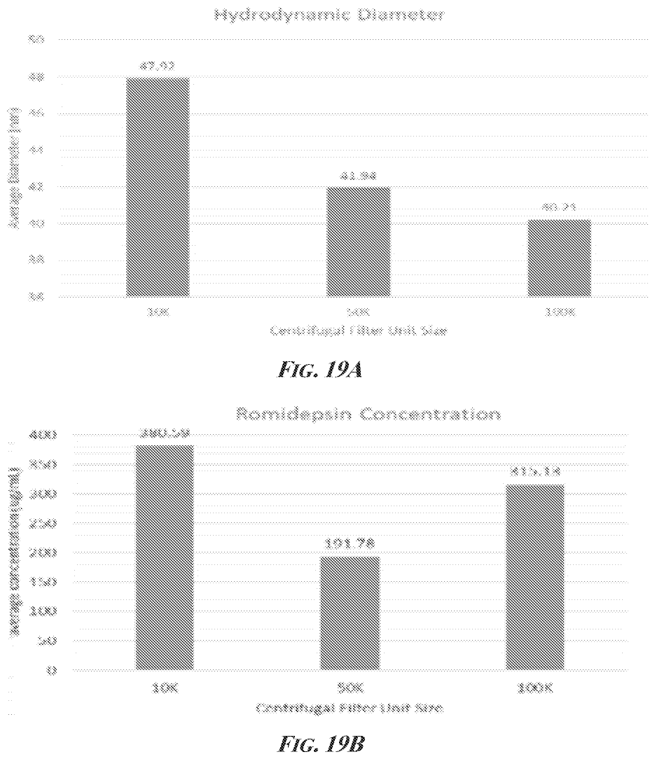

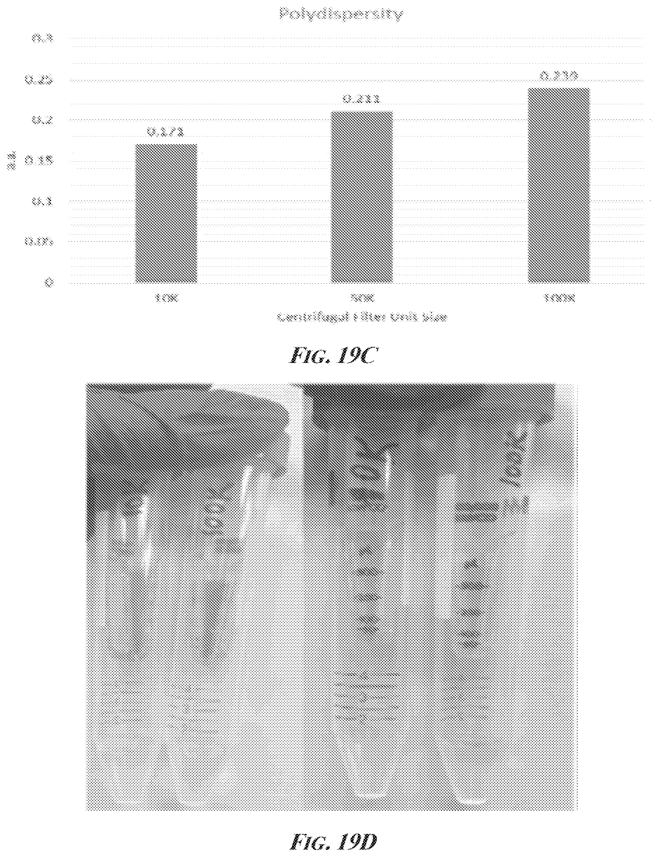

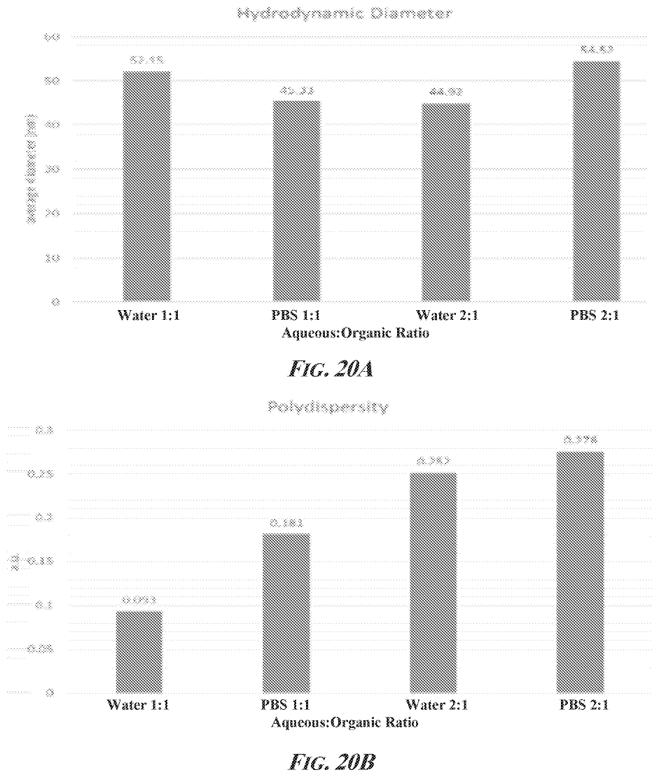

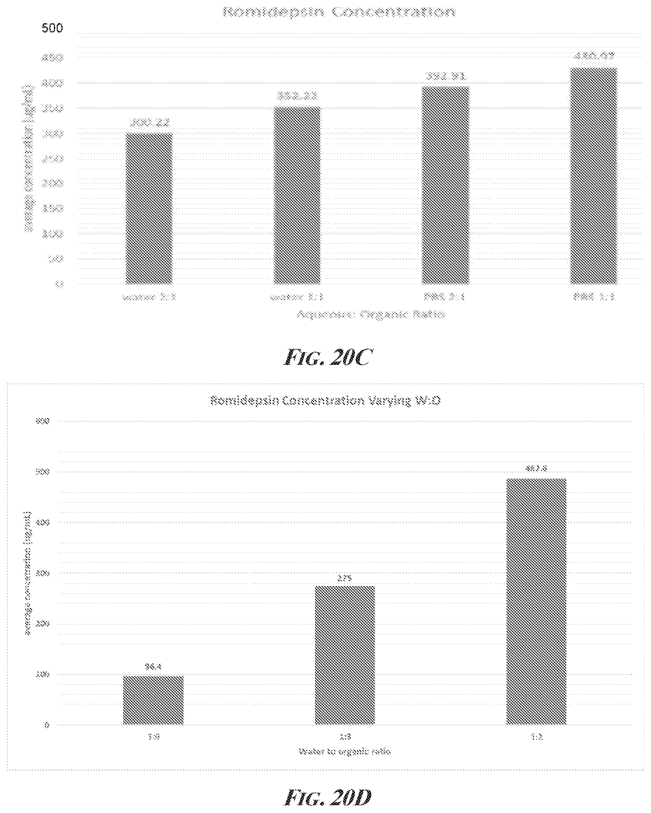

Additionally, the method reduces human error and time between experiments as all parameters are explored during the exact same synthesis. We elucidated the effect of the variables from each listed parameter, and their impact on the physio-chemical properties of the generated NPs. Solvent screening was conducted to test the romidepsin solubility in water miscible solvents including acetonitrile, acetone+10% DMSO, EtOH, and MeOH+Chloroform to test the NPs properties and drug loading. The surfactant, poloxamer 188 was added to aqueous phase to maintain the stability of the NPs and to avoid aggregation. We found acetonitrile is a desired solvent to dissolve romidepsin and m-PEG PDLLA as well as creating uniform sized particles with monodispersed formulation and higher encapsulation efficiency (FIGS. 17A-17C). While it is not desired to be bound by any particular method of operation, it might be related to the diffusion rate and viscosity of acetonitrile. This gives ‘tunability’ to NP physical characteristics during nanoprecipitation by modulating solvent properties. The polymer type (mPEG-PDLLA vs m-PEG PLGA), polymer length, ratio of hydrophilic PEG block to hydrophobic PDLLA block and drug to polymer ratio were included in this study to determine the parameter impact on NPs properties. This study may also be helpful in optimization of polymer to facilitate drug release. Polymer and drug were dissolved in acetonitrile, the desired solvent choice from solvent screening (iterative process). The data from this study clearly indicated that an optimal polymer was m-PEG PDLLA, 5K: 10K, and D/P ratio at 1/25 (W/W) because they have the highest impact on drug loading and size of the NPs (FIGS. 18A-18D). The centrifugal filters were used to process the prepared nanoparticle formulation to remove unencapsulated drug and residual solvent. We found Amicon 100K filters are more suitable for the given formulation with specific NP size in the range of 30-100 nM. We also showed co-encapsulation of Rhodamine B, a visualizing agent with Romidepsin in two different concentrations and used two different size filters to test the particles passage through the filters. The study demonstrated that excess drug flowed through the filters (deeper color tubes) and that the NP accommodates the co-drug (FIGS. 19A-19D). We finally tested the parameter that can significantly affect the drug loading into the particles and size of the particles, organic solvent addition into water and ratio of solvent to anti-solvent, as well as the solvent to 1×PBS as anti-solvent at 1:1 ratio with comparison to 1:1 water. The results are depicted in FIGS. 20A-20D. The most impactful variables on EE identified from this study are organic addition into water and solvent to the anti-solvent ratio of 1:1. We even found significantly higher encapsulation with 1×PBS.

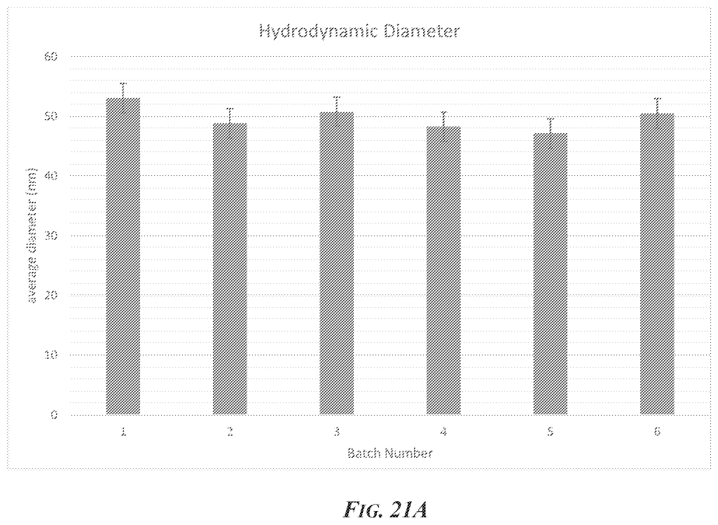

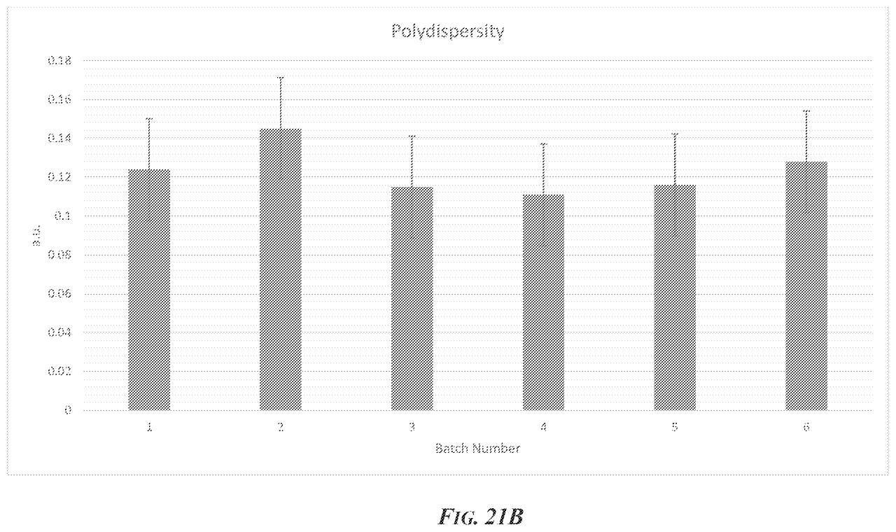

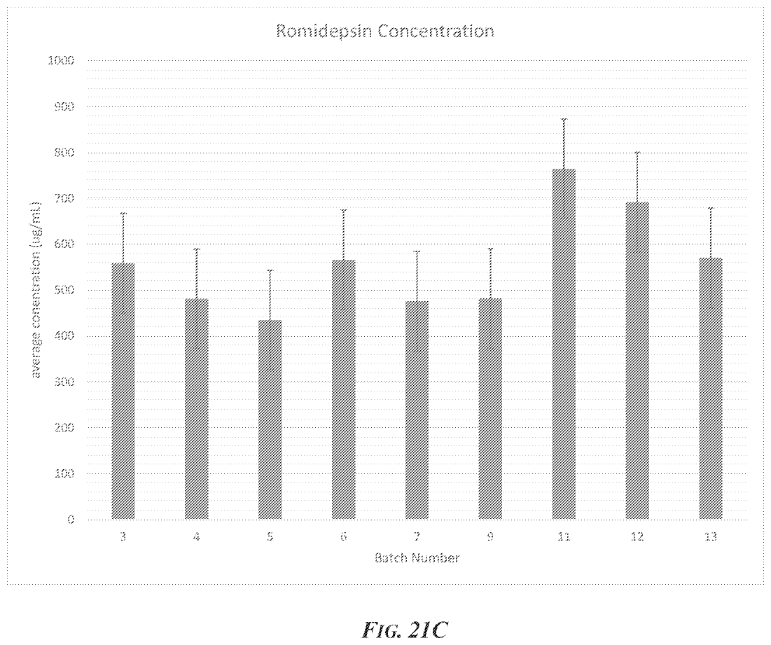

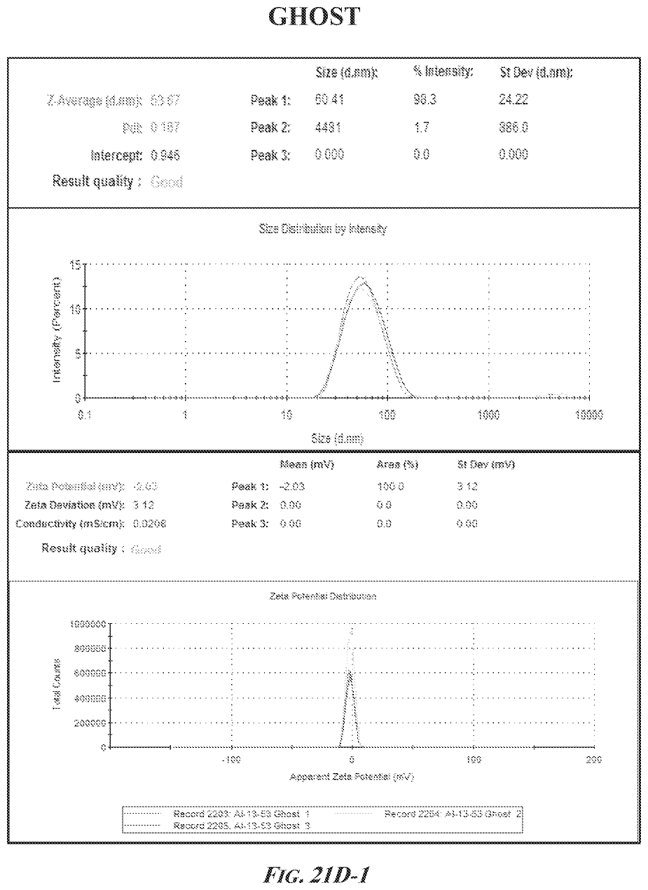

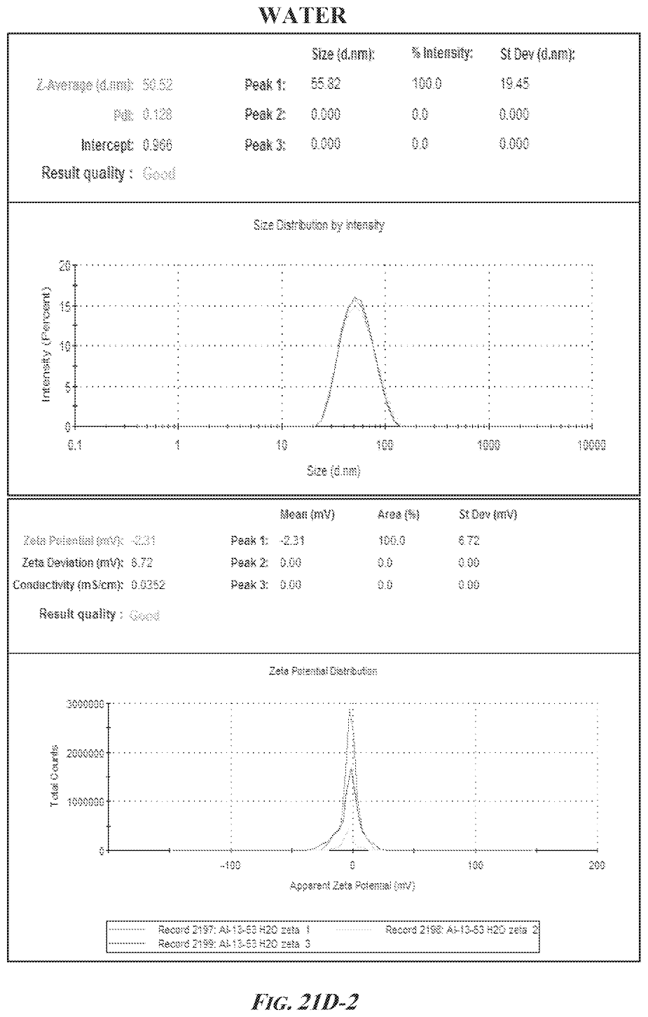

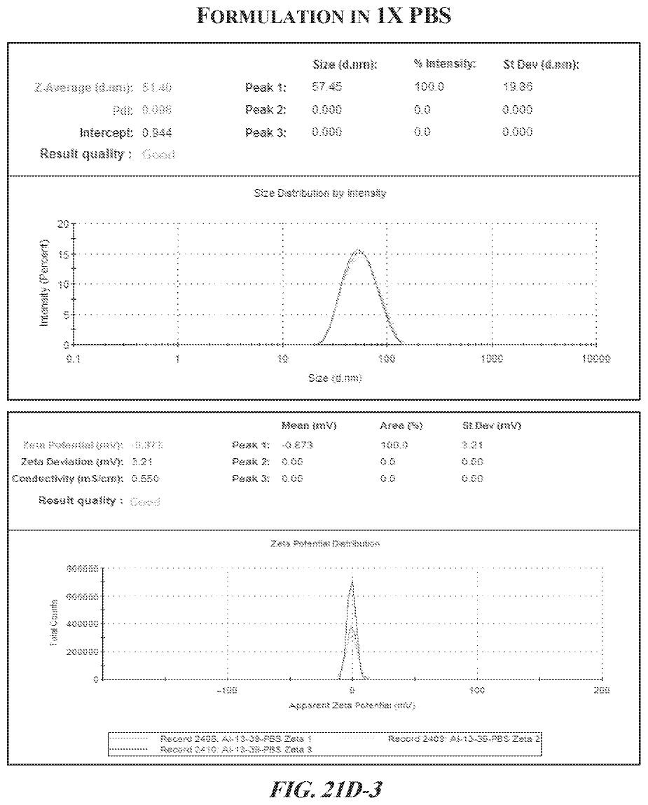

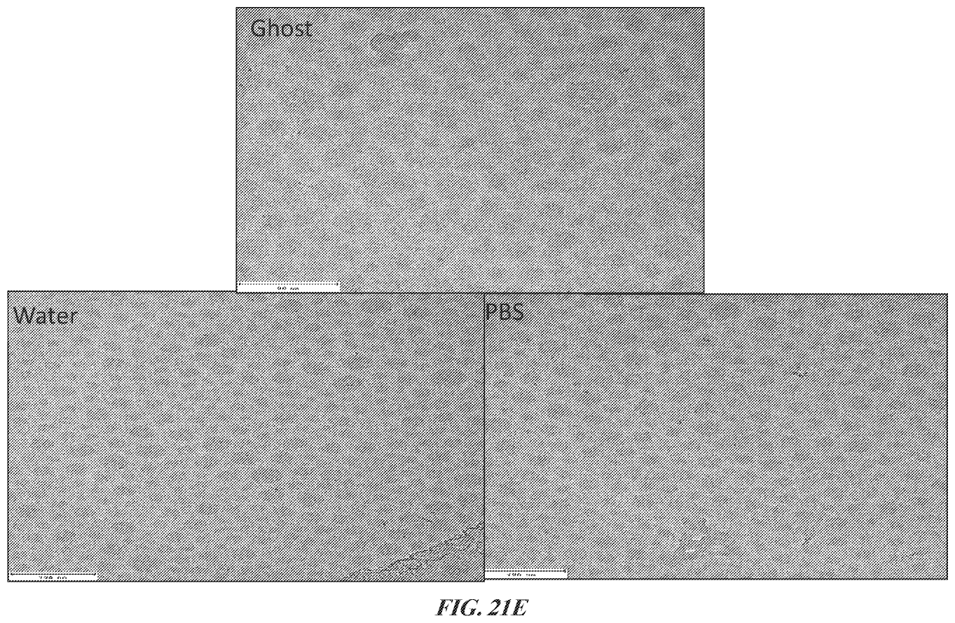

In summary, we found the importance of each parameter's role in NPs fabrication and properties that elucidate the interactions between parameters (Table 5B). We included a desired variable of all parameters from multiple optimizations, including: organic addition to water, D/P ratio, O/W ratio and rate of addition, to the scale-up process and fabricated multiple batches of NanoRomidepsin, 7-10 mL scale at 500 μg/mL concentration of romidepsin. The results clearly demonstrated batch-to-batch reproducibility with respect to size, PDI and drug loading in the particles. The volume of formulation scaled-up met the requirement of designed in vivo experiments. The size, PDI (DLS data), morphology of the NPs (cryo-EM), and drug concentration (LC/MS/MS) were depicted in FIGS. 21A-21E. See also Tables 5C, 5D, and 5E. Thus, the parallel approach of NPs physiochemical properties optimization is advantageous and improves the ability for the scale-up. This approach is an effective, controlled strategy for encapsulation of anticancer drugs and has the ability for scale-up. Theoretically, this synthesis approach could produce 300-400 mL of NanoRomidepsin with an EE of ˜50% without any further modifications in current laboratory set-up. The methodology that addresses the issues related to scale-up of Nano-sized drug formulations may facilitate the scope of commercialization.

### Discussion of the Examples

Romidepsin is the most potent HDAC inhibitor with greater inhibitory effects against the Class I HDACs, making it also the most selective of HDAC inhibitors. It has achieved two distinct regulatory approvals in the U.S. including: (1) full approval in patients with relapsed or refractory CTCL following one line of prior therapy in 2009, and (2) accelerated approval in patients with relapsed or refractory PTCL following one line of prior therapy in 2011. While in general the drug is well tolerated, it has been associated with a number of limitations, including: (i) potentially fatal cardiotoxicity; (ii) reactivation of EBV and hepatitis B viruses which has been associated with Grade 5 toxicity and a boxed warning; (iii) inconvenient schedule (administered over 4 hours weekly for 3 of 4 weeks); and a marginal benefit producing a response in only 25% of patients.

Despite these liabilities, the drug is associated with many clinically meaningful attributes, including: (i) durable durations of response in excess of a year; (ii) potent synergy with other epigenetically targeted drugs like DNMT inhibitors, and (iii) is the preferred HDAC inhibitors by treating physicians for patients with PTCL. In toto, strategies to reduce the romidepsin mediated toxicities and reduce the inconvenience of its schedule, coupled with efforts to improve tumor cell targeting and improved efficacy in combination, represent a valid approach to optimize this class of drugs not only for PTCL, but across other cancers and possibly autoimmune disorders.

The unique properties of nanoparticles, such as their small size, large surface-to-volume ratios, and the ability to achieve multivalency of targeting ligands on their surface, provide superior advantages for nanoparticle-based drug delivery to a variety of cancers. Nano-therapeutics in cancer target tumor cells through the carrier effect of nanoparticles and the positioning effect of the targeting substance after being absorbed.

The Nano polymer, Poly (D,L-lactic acid) (PDLLA) has a porous structure that exhibits better physicochemical properties, which is more convenient for adding anti-adhesion drugs. Thus, we developed a mPEG-PDLLA encapsulated romidepsin to reduce toxicity and provide a better therapeutic response in PTCL and autoimmune diseases. Our data to date have demonstrated the superiority of the nano-polymer derivatives over romidepsin as follows:
1. NanoRomidepsin PDLLA demonstrated cytotoxicity in CTCL, ALK-ALCL, LGL-leukemia and cutaneous melanoma cell lines with IC₅₀ values similar to unencapsulated romidepsin;
2. Treatment of PBMCs from healthy individuals with NanoRomidepsin PDLLA exhibited less cytotoxicity compared to PBMC treatment with romidepsin and less cytotoxicity compared to cancer lines therefore, has an increased therapeutic window;
3. NanoRomidepsin PDLLA induced apoptosis and increased acetylation of both histone H3 and H4 proteins. The change in acetylation of the histone proteins was 2 to 3-fold more in the NanoRomidepsin PDLLA treated cells compared to romidepsin.
4. NanoRomidepsin PDLLA showed equal or comparable toxicity to the free romidepsin in single and repeated dose toxicity assay. The maximum plasma concentration (Cmax), the integrated area under the plasma concentration-time curve (AUC) and the time of maximum plasma concentration (Tmax) derived from the pharmacokinetics analysis suggested that single dose of NanoRomidepsin administration exerted 3-4 fold higher exposure of romidepsin in plasma compared to the single dose of free romidepsin by intraperitoneal route. The Cmax, AUC, and Tmax indicated the nanoencapsulation of romidepsin results in long availability of romidepsin in plasma that would be effective to identify clinically relevant exposure in human.

As disclosed herein, the NanoRomidepsin PDLLA polymer was effective in targeting tumor cells, had lower cytotoxicity in normal cells, and had comparable/equal toxicity to romidepsin, and as such represents an asset with its own properties distinguished from romidepsin.

### REFERENCES

All references listed in the instant disclosure, including but not limited to all patents, patent applications and publications thereof, scientific journal articles, and database entries (including but not limited to GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Amengual et al. (2013) Sirtuin and pan-class I/II deacetylase (DAC) inhibition is synergistic in preclinical models and clinical studies of lymphoma. Blood 122:2104-13.
Bachy et al. (2020) Final Analysis of the Ro-CHOP Phase III Study (Conducted by LYSA): Romidepsin Plus CHOP in Patients with Peripheral T cell Lymphoma. Blood 136(Suppl 1):32-33.
Celgene (2021) Romidepsin: Investigator's Brochure (Edition 20.0) Release Date: 12 Apr. 2021.
Choudhary et al. (2009) Lysine acetylation targets protein complexes and co-regulates major cellular functions. Science 325(5942):834-40.
Falchi et al. (2021) Combined oral 5-azacytidine and romidepsin are highly effective in patients with PTCL: a multicenter phase 2 study. Blood 137(16):2161-2170.
Furumai et al. (2002) FK228 (depsipeptide) as a natural prodrug that inhibits class I histone deacetylases. Cancer Res. 62(17):4916-21.
Hoshino et al. (2007) Gene expression profiling induced by histone deacetylase inhibitor, FK228, in human esophageal squamous cancer cells. Oncol Rep 18(3):585-92.
Jain et al. (2015) Preclinical pharmacologic evaluation of pralatrexate and romidepsin confirms potent synergy of the combination in a murine model of human T cell lymphoma. Clin Cancer Res 21:2096-106.
Kakar et al. (2014) Hypersensitivity to romidepsin. J Am Acad Dermatol. 70(1):e21-2.
Kalac et al. (2011) HDAC inhibitors and decitabine are highly synergistic and associated with unique gene-expression and epigenetic profiles in models of DLBCL. Blood 118:5506-16.
Luchenko et al. (2014) Histone deacetylase inhibitor-mediated cell death is distinct from its global effect on chromatin. Mol Oncol. 8(8):1379-92.
Lue et al. (2016) Epigenetic Targeting with EZH2 and HDAC Inhibitors Is Synergistic in EZH2 Deregulated Lymphomas. Blood 128:839.
Marchi et al. (2015) The combination of hypomethylating agents and histone deacetylase inhibitors produce marked synergy in preclinical models of T cell lymphoma. British Journal of Haematology 171(2):215-226.
O'Connor et al. (2019) Oral 5-azacytidine and romidepsin exhibit marked activity in patients with PTCL: a multicenter phase 1 study. Blood 134(17):1395-1405.
Robertson et al. (1996) Characterization of a cell line, NKL, derived from an aggressive human natural killer cell leukemia. Exp Hematol 24:406-415.
Sasakawa et al. (2005) Marker genes to predict sensitivity to FK228, a histone deacetylase inhibitor. Biochem Pharmacol 69(4):603-616.
Scotto et al. (2021) Targeting the T cell Lymphoma Epigenome Induces Cell Death, Cancer Testes Antigens, Immune-Modulatory Signaling Pathways. Mol Cancer Ther 20(8):1422-1430.
Ueda et al. (1994) FR901228, a novel antitumor bicyclic depsipeptide produced by Chromobacterium violaceum No. 968. III. Antitumor activities on experimental tumors in mice. J Antibiot (Tokyo) 47(3):315-323.
U.S. Pat. No. 10,092,584.
Ververis et al. (2013) Histone deacetylase inhibitors (HDACIs): multitargeted anticancer agents. Biologics 7:47-60.

TABLE 1
FDA Approved Drugs Presently Used
in Patients with T Cell Lymphomas

| Approved Single | | | Outcomes | |
|---|---|---|---|---|
| Agent | | Mechanism | (ORR; CR; | Pivotal |
| Therapies | Indication | of Action | DOR) | Trial(s) |
| Belinostat | PTCL | HDACi | 26%; 11%; | (1) |
| FDA Approved | | | 13.6 months | |
| 2014 | | | | |
| Chidamide | PTCL | HDACi | 28%; 14%; | (2) |
| Approved only | | | 9.9 months | |
| in China | | | | |
| Brentuximab | ALCL | α-CD30 linked | 86%; 57%; | (3, 4) |
| vedotin FDA | | to auristatin | 13.2 months | |
| Approved 2011 | | (antitubulin | | |
| for ALCL | | agent) | | |
| Pralatrexate | PTCL | DHFR/ | 29%; 11%; | (5) |
| FDA Approved | | thymidylate | 10.1 months | |
| 2009 | | synthase inhibitor | | |
| Romidepsin | PTCL | HDACi | 25%; 15%; | (6) |
| FDA Approved | | | 28 months | |
| 2011 | | | 38%; 18%; | (7) |
| | | | 8.9 months | |

(1) O'Connor et al., 2015;
(2) Shi et al., 2015;
(3) Pro et al., 2012;
(4) Horwitz et al., 2018;
(5) O'Connor et al., 2011;
(6) Coiffier et al., 2014;
(7) Piekarz et a., 2011.

TABLE 2
Summary of IC₅₀ Values at 60 Hours for All Cancer
Cell Lines Tested in Response to Treatment with
Romidepsin or the Three Nanopolymers

| | IC₅₀ in nM at 60 Hours | | | |
|---|---|---|---|---|
| | | NanoRomi | NanoRomi | NanoRomi |
| | | m-PEG | m-PEG | m-PEG |
| Cell | | PDLLA | PDLLA | PLGA |
| Line | Romidepsin | PBS | H₂O | PBS |
| SUP-T1 | 1.9 | 2.3 | 1.9 | 2.6 |
| FEPD | 1.6 | 1.8 | 1.7 | 3.3 |
| H9 | 1.4 | 4.6 | 1.3 | 5.5 |
| HH | 1.3 | 7.5 | 1 | 1.7 |
| SUP-M2 | 1.6 | 2 | 1.4 | 4.6 |
| NKL | 0.7 | 1.7 | 1.1 | 2.3 |
| TL-1 | 0.7 | 1.3 | 1 | 1.9 |
| FM3-29 | 0.6 | 4.3 | 0.7 | 1.1 |

IC₅₀ values were determined based on cell line and time using GraphPad Prism software.

TABLE 3
Summary of IC₅₀ Calues at 24, 48, and 72
Hours for PBMCs from Healthy Donors Treated
with Romidepsin or NanoRomidepsin PDLLA

IC50 in nM

| | Time | Nano-Romi m-PEG | |
|---|---|---|---|
| PBMC | (hrs) | PDLLA H20 | Romidepsin |
| Donor 1 | 24 | 16.40 | 0.02 |
| | 48 | 0.45 | 0.01 |
| | 72 | 6.32 | 0.04 |
| Donor 2 | 24 | >50 | >50 |
| | 48 | >50 | 0.01 |
| | 72 | 0.43 | 0.07 |
| Donor 3 | 24 | >50 | >50 |
| | 48 | 11.24 | >50 |
| | 72 | 2.20 | 2.48 |

TABLE 4
Pharmacokinetic Parameters for Free Romidepsin and NanoRomi
After Single Dose Intraperitoneal or Intravenous Treatment

| | |
|---|---|
| Free Romi | NanoRomi |

| | | |
|---|---|---|
| Parameter | Unit | Intraperitoneal |

| Lambda_z | l/h | 0.07 | 0.06 |
|---|---|---|---|
| t½ | h | 9.83 | 11.69 |
| Tmax | h | 6.00 | 3.00 |
| Cmax | ng/ml | 119.90 | 434.71 |
| Tlag | h | 0.00 | 0.00 |
| Clast_obs/Cmax | | 0.01 | 0.01 |
| AUC 0-t | ng/ml*h | 1918.97 | 6939.90 |
| AUC 0-inf_obs | ng/ml*h | 1929.03 | 7035.71 |
| AUC 0-t/0-inf_obs | | 0.99 | 0.99 |
| AUMC 0-inf_obs | ng/ml*h² | 24602.73 | 117777.85 |
| MRT 0-inf_obs | h | 12.75 | 16.74 |
| Vz/F_obs | (mg)/(ng/ml) | 0.02 | 0.01 |
| Cl/F_obs | (mg)/(ng/ml)/h | 0.00 | 0.00 |
| | | Free Romi | NanoRomi |

| | | |
|---|---|---|
| Parameter | Unit | Intravenous |

| | | | |
|---|---|---|---|
| Lambda_z | l/h | 0.13 | 0.09 |
| t½ | h | 5.22 | 7.63 |
| Tmax | h | 1.00 | 1.00 |
| Cmax | ng/ml | 21.31 | 231.02 |
| T_infusion | h | 1.50 | 1.50 |
| Clast_obs/Cmax | | 0.06 | 0.01 |
| AUC 0-t | ng/ml*h | 99.27 | 2532.12 |
| AUC 0-inf_obs | ng/ml*h | 108.48 | 2569.62 |
| AUC 0-t/0-inf_obs | | 0.92 | 0.99 |
| AUMC 0-inf_obs | ng/ml*h² | 681.51 | 29353.59 |
| MRT 0-inf_obs | h | 5.53 | 10.67 |
| Vz_obs | (mg)/(ng/ml) | 57.23 | 3.53 |
| Cl_obs | (mg)/(ng/ml)/h | 7.60 | 0.32 |

TABLE 5A
Optimization of Encapsulation Efficiency of Romidepsin

| | Solvent/ | | | | | |
|---|---|---|---|---|---|---|
| Organic phase | Non- | Stabilizing | | | | |
| Drug/Polymer | solvent | agent in | | Rate of | Drug | Encapsulation |
| (m-PEG | (O/W | water | Order of | addition | loading | efficiency |
| PDLLA):D/P | ratio) | Poloxamer | addition | (mL/hour) | (ug/mL) | (% EE) |
| 1 mg/10 mg | 1 to 9 | None | Water to | 30 | 56 | 1.4 |
| (5K:10K) + | | | Organic | | | |
| 10% surfactant | | | | | | |
| 1 mg/5 mg | 1 to 9 | 10 | Organic | 30 | 28.0 | 2.1 |
| (5K:10K) | | | to water | | | |
| 1 mg/10 mg | 1 to 9 | 10 | Organic | 30 | 42.7 | 3.2 |
| (5K:10K) | | | to water | | | |
| 1 mg/25 mg | 1 to 9 | 10 | Organic | 30 | 92.4 | 11.8 |
| (5K:10K) | | | to water | | | |
| 1 mg/50 mg | 1 to 9 | 10 | Organic | 30 | 96.4 | 9.6 |
| (5K:10K) | | | to water | | | |
| 1 mg/10 mg | 1 to 3 | 10 | Organic | 30 | 131.3 | 13.1 |
| (5K:10K) | | | to water | | | |
| 1 mg/25 mg | 1 to 3 | 10 | Organic | 30 | 275.0 | 27.5 |
| (5K:10K) | | | to water | | | |
| 1 mg/25 mg | 1 to 2 | 10 | Organic | 30 | 487.6 | 48.76 |
| (5K:10K) | | | to water | | | |
| 1 mg/25 mg | 1 to 2 | 20 | Organic | 20 | 349.0 | 34.9 |
| (5K:10K) | | | to water | | | |
| 1 mg/25 mg | 1 to 2 | 5 | Organic | 30 | 552.3 | 55.23 |
| (5K:10K) | | | to water | | | |
| 1 mg/25 mg | 1 to 2 | 2.5 | Organic | 30 | 444.3 | 44.43 |
| (5K:10K) | | | to water | | | |
| 1 mg/25 mg | 1 to 2 | 10 | Organic | 50 | 762.6 | 76.26 |
| (5K:10K) | | | to water | | | |

TABLE 5B
Parameter Optimization for Scale-up to Enhance
Encapsulation Efficiency of Romidepsin

| | | Stabilizing | | | |
|---|---|---|---|---|---|
| (m-PEG | Solvent/ | agent in | | Rate of | Drug |
| PDLLA):D/P | Nonsolvent | water | Way of | addition | loading |
| 1/25 | (O/W ratio) | Poloxamer | addition | (mL/hour) | (ug/mL) |
| Drug in | 1 to 2 | 10 | Organic | 50 | 155.99 |
| Acetone + | | | to water | | |
| 10% DMSO | | | | | |
| Drug in ACN | 1 to 2 | 10 | Organic | 50 | 323.24 |
| | | | to water | | |
| Drug in THF | 1 to 2 | 10 | Organic | 50 | 186.96 |
| | | | to water | | |
| Drug in ETOH | 1 to 2 | 10 | Organic | 50 | 214.23 |
| | | | to water | | |
| 1 mg/25 mg | 1 to 2 | 10 | Organic | 50 | 202.88 |
| (5K:4K) | | | to water | | |
| 1 mg/25 mg | 1 to 2 | 10 | Organic | 50 | 300.22 |
| (5K:10K) | | | to water | | |
| 1 mg/25 mg | 1 to 1 | 10 | Organic | 50 | 352.22 |
| (5K:10K) | | | to water | | |
| 1 mg/25 | 1 to 2 | 10 | Organic | 50 | 200.62 |
| mg (5K:14K) | | | to water | | |
| 1 mg/25 mg | 1 to 2 | 10 | Organic | 50 | 221.14 |
| (5K:20K) | | | to water | | |
| mPEG-PLGA | 1 to 2 | 10 | Organic | 50 | 149.29 |
| (5K:20K) | | | to water | | |
| 10K MWCO | 1 to 2 | 10 | Organic | 50 | 380.59 |
| filter | | | to water | | |
| 50K MWCO | 1 to 2 | 10 | Organic | 50 | 223.23 |
| filter | | | to water | | |
| 100K MWCO | 1 to 2 | 10 | Organic | 50 | 315.13 |
| filter | | | to water | | |

TABLE 5C
Summary of Exemplary Scaled-up Batches of NanoRomidepsin

| Batch | | D_{H} | | Drug Conc. | EE |
|---|---|---|---|---|---|
| Number | Name | (nm) | PDI | (ug/mL) | (%) |
| 1 | AI-13-39 | 53.07 | 0.124 | 490.6075 | 34 |
| 2 | AI-13-41-H₂O | 48.84 | 0.145 | 559.0933333 | 50.1 |
| 3 | AI-13-45-H₂O | 50.76 | 0.115 | 565.6333333 | 39.6 |
| 4 | AI-13-49-40 min | 48.25 | 0.111 | 481.925 | 48.1 |
| 5 | AI-13-51 | 47.14 | 0.116 | 382.23625 | 41.8 |
| 6 | AI-13-53-H₂O | 50.52 | 0.128 | 570.35 | 39.90 |

TABLE 5D
Comparison of Physical Properties of Ghost and
NanoRomidepsin Particles in Water and PBS

| NP | Z-Average (nm) | PDI | Z-potential (mV) |
|---|---|---|---|
| Ghost in Water | 53.67 | 0.167 | −2.31 |
| In water | 50.52 | 0.128 | −2.03 |
| In PBS | 51.40 | 0.098 | −0.873 |

TABLE 5E
Average of Multiple Batches Concentration and Size of Particles

| | Z- | | | Encapsulation |
|---|---|---|---|---|
| | Average | | Drug Conc. | Efficiency |
| n = 14 | (nm) | PDI | (μg/mL) | (%) |
| Average | 49.76 | 0.123 | 508.31 | 42.25 |
| Standard | 2.12 | 0.012 | 72.96 | 5.94 |
| Deviation | | | | |

1. A composition comprising a histone deacetylase inhibitor (HDACi) encapsulated in and/or otherwise associated with a nanoparticle, optionally wherein HDACi is selected from the group consisting of vorinostat, romidepsin, belinostat, and panobinostat, or any combination thereof, further optionally wherein the HDACi is romidepsin.

2. (canceled)

3. The composition of claim 1, comprising one or more polymers and/or one or more surfactants, optionally wherein the one or more polymers are selected from the group consisting of a polyester, further optionally PDLLA, PLGA, PLA, and/or PCL, copolymers thereof, and blends thereof.

4. (canceled)

5. The composition of claim 3, wherein the polymer comprises a polymer selected from the group consisting of a synthetic polymer; a biodegradable polymer; a biocompatible polymer; an amphiphilic polymer; a diblock co-polymer; and blends thereof.

6. The composition of claim 3, wherein the polymer comprises a hydrophilic, PEG chain, optionally methoxy PEG, PEG-carboxylic acid, PEG-hydroxyl, and/or PEG amine as end cap and chain length range 2K-10K.

7. The composition of claim 3, wherein the polymer is a hydrophobic core-forming polymer, optionally a hydrophobic core-forming polymer selected from the group consisting of PDLLA, PLGA, PLA, and/or PCL.

8. The composition of claim 1, where in the nanoparticle comprises a methyl ether-PEG polylactide-co-glycolide (mPEG-PLGA,50:50).

9. The composition of claim 1, wherein one or more parameters selected from a group consisting of mode of phase addition, HDACi/polymer ratio, HDACi/surfactant ratio, solvent/anti-solvent ratio, rate of addition, and combinations thereof are optimized.

10. The composition of claim 9, wherein:
(a) the HDACi/polymer ratio ranges from about 1:10 to about 1:100 W/W, optionally 1:10 to about 1:50 W/W;
(b) the HDACi/surfactant ratio ranges from about 1:0.05 to about 1:0.2 W/W;
(c) the solvent/anti-solvent ratio ranges from about 1:10 to about 1:1, optionally wherein the anti-solvent is selected from the group consisting of water, PBS, or another ionic buffer solution; and/or
(d) the rate of addition ranges from about 10 to about 500 mL/hour, optionally about 10 to about 50 mL/hour.

11. A method for treating a disease, disorder, or condition associated with sensitivity to a histone deacetylase inhibitor (HDACi), the method comprising administering to a subject in need thereof an effective amount of the composition of claim 1.

12. The method of claim 11, wherein the disease, disorder, or condition associated with sensitivity to an HDACi is a tumor and/or a cancer, an inflammatory disease, disorder, or condition; an autoimmune disease, disorder, or condition; or any combination thereof, optionally wherein the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

13. (canceled)

14. A method for inhibiting the growth, proliferation, and/or metastasis of a tumor and/or a cancer associated with sensitivity to a histone deacetylase inhibitor (HDACi), the method comprising administering to a subject in need thereof an effective amount of the composition of claim 1, optionally wherein the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

15. (canceled)

16. The method of claim 14, further comprising administering to the subject at least one additional therapeutically active agent.

17. The method of claim 16, wherein the at least one additional therapeutically active agent is a chemotherapeutic agent, optionally wherein the chemotherapeutic agent is selected from the group consisting of cyclophosphamide, doxorubicin; vincristine, prednisone, azacytidine, decitabine, cladribine, methotrexate, pralatrexate, and cyclosporin A, and combinations thereof.

18. (canceled)

19. A method for treating an inflammatory and/or an autoimmune disease, disorder, or condition associated with sensitivity to a histone deacetylase inhibitor (HDACi), the method comprising administering to a subject in need thereof an effective amount of the composition of claim 1.

20. The method of claim 19, wherein the inflammatory and/or an autoimmune disease, disorder, or condition is selected from the group consisting of fatty liver disease, endometriosis, types 1 and 2 diabetes, inflammatory bowel disease, asthma, obesity, Alzheimer's and Parkinson's diseases, Ankylosing Spondylitis (AS), Antiphospholipid Antibody Syndrome (APS), Gout, Inflammatory Arthritis Center, Myositis, Rheumatoid Arthritis, Scleroderma, Sjogren's Syndrome, Systemic Lupus Erythematosus (SLE, Lupus), vasculitis, Addison's disease, Celiac disease-sprue (gluten-sensitive enteropathy), dermatomyositis, Grave's disease, Hashimoto's thyroiditis, Multiple sclerosis, Myasthenia gravis, Pernicious anemia, Reactive arthritis, Psoriasis/psoriatic arthritis, multiple sclerosis, Systemic lupus erythematosus (SLE), type 1 diabetes, Inflammatory bowel disease (including Crohn's disease and ulcerative colitis), autoimmune vasculitis, Guillain-Barre syndrome, and Chronic inflammatory demyelinating polyneuropathy.

21. The method of claim 19, further comprising administering to the subject at least one additional therapeutically active agent, optionally wherein the at least one additional therapeutically active agent is an anti-inflammatory and/or an immunosuppressant agent.

22. (canceled)

23. A method of fabricating a nanoparticle comprising a drug molecule, the method comprising:
(a) varying in one or more iterations two or more parameters of a first or subsequent reaction mixture comprising a drug molecule and one or more polymers;
(b) selecting a desired combination of parameters for a further reaction mixture based on the varying of step (a); and
(c) precipitating a nanoparticle comprising the drug molecule from the further reaction mixture.

24. The method of claim 23, wherein the reaction mixture further comprises a reaction mixture selected from the group consisting of a solvent, a non-solvent, a surfactant, and combinations thereof, optionally wherein the solvent is an organic solvent and/or the non-solvent is an aqueous solvent, water, or PBS buffer.

25. (canceled)

26. (canceled)

27. The method of claim 23, comprising optimizing one or more parameters selected from a group consisting of mode of phase addition, a drug/polymer ratio, a drug/surfactant ratio, solvent/anti-solvent ratio, rate of addition, and combinations thereof, optionally wherein the drug is a histone deacetylase inhibitor (HDACi), further optionally romidepsin.

28. (canceled)

29. The method of claim 28, wherein:
(a) the HDACi/polymer ratio ranges from about 1:10 to about 1:100 W/W, optionally 1:10 to about 1:50 W/W;
(b) the HDACi/surfactant ratio ranges from about 1:0.05 to about 1:0.2 W/W;
(c) the solvent/anti-solvent ratio ranges from about 1:10 to about 1:1, optionally wherein the anti-solvent is selected from the group consisting of water, PBS, or another ionic buffer solution; and/or
(d) the rate of addition ranges from about 10 to about 500 mL/hour, optionally about 10 to about 50 mL/hour.
